(19) United States
(12) Patent Application Publication
(10) Pub. No.: US 2023/0250262 A1
(43) Pub. Date: Aug. 10, 2023

(54) CELLULOSIC PARTICLE

(71) Applicant: FUJIFILM Business Innovation Corp., Tokyo (JP)

(72) Inventors: Kenji YAO, Kanagawa (JP); Hideaki YOSHIKAWA, Kanagawa (JP); Takahiro ISHIZUKA, Kanagawa (JP); Hirokazu HAMANO, Kanagawa (JP); Tetsuya TAGUCHI, Kanagawa (JP); Masahiro OKI, Kanagawa (JP); Yuko IWADATE, Kanagawa (JP); Kazusei YOSHIDA, Kanagawa (JP); Ayu NAITO, Kanagawa (JP)

(73) Assignee: FUJIFILM Business Innovation Corp., Tokyo (JP)

(21) Appl. No.: 17/990,720

(22) Filed: Nov. 20, 2022

(30) Foreign Application Priority Data
Feb. 8, 2022 (JP) 2022-017988

(51) Int. Cl. C08L 1/12; C08K 5/20; C08K 5/13; C08J 3/12

(52) U.S. Cl. CPC C08L 1/12; C08K 5/20; C08K 5/13; C08J 3/128; C08J 2301/12

(57) ABSTRACT

A cellulosic particle contains a first component that is cellulose; and a second component that is at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C).

### CROSS-REFERENCE TO RELATED APPLICATIONS

This application is based on and claims priority under 35 USC 119 from Japanese Patent Application No. 2022-017988 filed Feb. 8, 2022.

### BACKGROUND

### (i) Technical Field

The present disclosure relates to a cellulosic particle.

### (ii) Related Art

In Japanese Unexamined Patent Application Publication No. 2020-132616, “oily solid cosmetics containing surface-treated spherical cellulose powder with an average particle size of 1.0-30.0 μm.” are proposed.

In Japanese Patent No. 6872068, “resin beads formed of a resin containing cellulose as a main component, wherein the particle size at a cumulative percentage of 50% in terms of volume is 50 μm or less, the sphericity is 0.7-1.0, the surface smoothness is 70-100%, the solidity is 50-100%, the five-day biodegradability measured according to JIS K6950:2000 (ISO 14851:1999) is 20% or greater, and the content of cellulose in the resin is 90-100 mass %.” are proposed.

### SUMMARY

Aspects of non-limiting embodiments of the present disclosure relate to a particle containing cellulose, or a cellulosic particle, that may be superior in biodegradability and flexibility compared with if not containing at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C).

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided a cellulosic particle containing: a first component that is cellulose; and a second component that is at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C).

### DETAILED DESCRIPTION

Exemplary embodiments of the present disclosure will now be described. The following description and the Examples are for illustrating exemplary embodiments and do not limit the scope of aspects of the present disclosure.

In a series of numerical ranges presented herein, the upper or lower limit of a numerical range may be substituted with that of another in the same series. The upper or lower limit of a numerical range, furthermore, may be substituted with a value indicated in the Examples section.

A constituent may be a combination of multiple substances.

If a composition contains a combination of multiple substances as one of its constituents, the amount of the constituent represents the total amount of the substances in the composition unless stated otherwise.

### Cellulosic Particles

Cellulosic particles according to an exemplary embodiment contain a first component that is cellulose; and a second component that is at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C).

Configured as described above, the cellulosic particles according to this exemplary embodiment may be superior in biodegradability and flexibility. A possible reason is as follows.

Particles containing cellulose (hereinafter referred to as cellulosic particles) may be advantageous in that they may be highly biodegradable by virtue of containing cellulose. Cellulosic particles, however, tend to be hard because of containing cellulose, and this has limited their applications.

Making cellulosic particles with cellulose and a non-cellulose ingredient may help render the cellulosic particles flexible. In such attempts, however, the cellulose tends to be of low miscibility with the non-cellulose ingredient.

To address this, the cellulosic particles according to this exemplary embodiment are made to contain not only a first component that is cellulose but also a second component highly flexible compared with cellulose (specifically, at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C)). Although not certain, it appears that such cellulosic particles tend to form a sea-island structure composed of a sea portion formed by the first component and island portions formed by the second component inside themselves. The first and second components, furthermore, are of low miscibility with each other; the region of the island portions, formed by the second component, therefore, tends to be large in size. In that case the flexibility of the region of the island portions tends to have great impact on the flexibility of the cellulosic particles. For this reason, presumably, the cellulosic particles according to this exemplary embodiment may be superior in flexibility.

The cellulosic particles according to this exemplary embodiment, furthermore, may also be superior in biodegradability by virtue of containing cellulose.

For this reason, presumably, the cellulosic particles according to this exemplary embodiment, configured as described above, may be superior in biodegradability and flexibility.

### First Component

The cellulosic particles according to this exemplary embodiment contains a first component that is cellulose.

The number-average molecular weight of the cellulose may be 37000 or more, preferably 45000 or more.

There is no particular upper limit to the number-average molecular weight of the cellulose, but for example, the number-average molecular weight may be 100000 or less.

Making the number-average molecular weight of the cellulose 37000 or more may tend to make the cellulosic particles better in flexibility. A possible reason is as follows.

As the molecular weight of the cellulose increases, the number of terminal hydroxyl groups decreases, and the number of hydrogen bonds formed at the termini decreases accordingly. This may help impart flexibility to the rigid molecular chains of the cellulose by preventing them from growing too long.

For this reason, presumably, the cellulosic particles may tend to be better in flexibility.

Making the number-average molecular weight of the cellulose 37000 or more, furthermore, may make it more likely that the cellulosic particles are highly biodegradable and hardly change its flexibility over time.

Making the number-average molecular weight of the cellulose 37000 or more may be an easy way to limit the initial rate of biodegradation of the particles. This may be an easy way to limit the destruction of the surface of the cellulosic particles or deformation of the cellulosic particles caused by biodegradation; the change in flexibility over time, therefore, may become smaller. Limiting the initial rate of biodegradation may also encourage the improvement of biodegradability because it may make the collapse of the cellulosic particles caused by the biodegradation of the cellulosic particles nearly uniform.

The number-average molecular weight of the cellulose is measured by gel permeation chromatography (differential refractometer, Optilab T-rEX, Wyatt Technology; multiangle light scattering detector, DAWN HELEOS II, Wyatt Technology; columns, one TSKgel α-M and one α-3000, Tosoh) with dimethylacetamide eluent (containing 0.1 M lithium chloride).

The amount of the first component may be 70% by mass or more and 95% by mass or less of the total amount of the first and second components.

Ensuring that the amount of the first component falls within this range may tend to make the cellulosic particles better in biodegradability and flexibility. A possible reason is as follows.

Making the amount of the first component 70% by mass or more of the total amount of the first and second components means making the cellulosic particles rich in cellulose. This may encourage further improvement of the biodegradability of the cellulosic particles.

Making the amount of the first component 95% by mass or less of the total amount of the first and second components, furthermore, may help ensure that the cellulose content of the cellulosic particles is not too large. This may help limit the associated decrease in the flexibility of the cellulosic particles.

For this reason, presumably, ensuring that the amount of the first component falls within the above range may tend to make the cellulosic particles better in biodegradability and flexibility.

In order for the cellulosic particles to be even better in biodegradability and flexibility, the amount of the first component may be 75% by mass or more and 90% by mass or less, preferably 80% by mass or more and 85% by mass or less, of the total amount of the first and second components.

In order for the cellulosic particles to be superior in biodegradability and flexibility, the amount of the first component may be 75% by mass or more and 90% by mass or less, preferably 80% by mass or more and 90% by mass or less, more preferably 85% by mass or more and 90% by mass or less of the cellulosic particles as a whole.

If the cellulosic particles have a coating layer as described later herein, however, the amount of the first component represents the amount in the core particle, which has the coating layer formed thereon and contains the first and second components, as a whole.

### Second Component

The cellulosic particles according to this exemplary embodiment contain a second component that is at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C).

### Fatty Acid Derivative (A)

A fatty acid derivative (A) is a compound obtained by allowing the carboxy group in a fatty acid to react with another functional group.

Examples of functional groups include the amino group and the hydroxy group.

Examples of fatty acid derivatives (A), therefore, include fatty acid amides and fatty acid esters.

In this context, “fatty acid” refers to a compound that can be expressed with the general formula CₙHₘCOOH (n and m are integers).

The fatty acid derivative (A) may be a fatty acid derivative having a saturated aliphatic group with 10 or more and 25 or fewer carbon atoms (C10 to C25), preferably a fatty acid derivative having a C12 to C20 saturated aliphatic group, more preferably a fatty acid derivative having a C14 to C18 saturated aliphatic group.

Using a fatty acid derivative having a C10 to C25 saturated aliphatic group as a fatty acid derivative (A) may tend to make the cellulosic particles better in flexibility. A possible reason is as follows.

Fatty acid derivatives having a C10 to C25 saturated aliphatic group tend to be highly miscible with cellulose acylates, which can be used as raw materials in the production of cellulosic particles. Using such a fatty acid derivative as a fatty acid derivative (A), therefore, may encourage the formation of a sea-island structure composed of a sea portion formed by the first component and island portions formed by the second component inside the cellulosic particles, and the region of the island portions, formed by the second component, may tend to grow to a larger size accordingly.

For this reason, presumably, the cellulosic particles may tend to be better in flexibility.

The octanol/water partition coefficient (hereinafter also referred to as the O/W coefficient) of the fatty acid derivative having a C10 to C25 saturated aliphatic group may be 5 or greater and 10 or less, preferably 6 or greater and 9 or less, more preferably 7 or greater and 8 or less.

In this context, the octanol/water partition coefficient is a value calculated through the following.

A sample is dissolved in a mixture of octanol and water in a ratio by mass of 1/1, and the concentration of the substance in the octanol (Co) and the concentration of the substance in the water (Cw) are measured; then the octanol/water partition coefficient (O/W coefficient) is calculated according to equation (1) below.

O/W coefficient=Log(Co/Cw) (1)

(where “Log” means that the term is a common logarithm)

The concentration of the substance in the octanol (Co) and the concentration of the substance in the water (Cw) are measured as follows.

The OECD test guideline is followed. More specifically, a sample is dissolved in a mixture of 1-octanol and water in a ratio by mass of 1/1, and the resulting solution is centrifuged until it completely separates into two layers. From the 1-octanol layer, an appropriate amount of octanol solution is pipetted. The tip of a syringe, into which air has been drawn in advance, is inserted into the water layer through the 1-octanol layer while the air is ejected, and the aqueous solution is sampled quickly.

For each solution, the test concentration is determined using an ion chromatograph (Metrohm, 930 Compact IC).

If the octanol/water partition coefficient (O/W coefficient) of the fatty acid derivative having a C10 to C25 saturated aliphatic group is 5 or greater and 10 or less, the cellulosic particles may tend to be better in flexibility. A possible reason is as follows.

Fatty acid derivatives having a C10 to C25 saturated aliphatic group and an O/W coefficient of 5 or greater and 10 or less tend to be highly miscible with cellulose acylates, which can be used as raw materials in the production of cellulosic particles. Using such a fatty acid derivative as a fatty acid derivative (A), therefore, may encourage the formation of a sea-island structure composed of a sea portion formed by the first component and island portions formed by the second component inside the cellulosic particles, and the region of the island portions, formed by the second component, may tend to grow to a larger size accordingly.

For this reason, presumably, the cellulosic particles may tend to be better in flexibility.

For higher flexibility of the cellulosic particles, the fatty acid derivative (A) may be a fatty acid amide.

The fatty acid amide may be a fatty acid amide obtained by amidating a fatty acid and an amine.

For higher flexibility of the cellulosic particles, the fatty acid used to synthesize the fatty acid amide may be a saturated fatty acid, preferably a C10 to C25 saturated fatty acid, more preferably a C15 to C20 saturated fatty acid, even more preferably octacosanoic acid.

Examples of amines used to synthesize the fatty acid amide include primary amines and secondary amines.

For higher flexibility of the cellulosic particles, the amine used to synthesize the fatty acid amide may be an amine having one or more hydroxy groups (hereinafter also referred to as an aminoalcohol).

For higher flexibility of the cellulosic particles, the amine used to synthesize the fatty acid amide may have a structure composed of a divalent hydrocarbon group and amino and hydroxy groups bound thereto.

The divalent hydrocarbon group may be a C1 to C10 one, preferably a C2 to C5 one.

Examples of ethanolamines used to synthesize the fatty acid amide include methanolamine, ethanolamine, 3-amino-1-propanol, 4-amino-1-butanol, and diethanolamine.

The fatty acid derivative (A) may be a fatty acid amide obtained by amidating a fatty acid and an aminoalcohol (hereinafter also referred to as a fatty acid ethanolamide).

Using a fatty acid ethanolamide as a fatty acid derivative (A) may tend to make the cellulosic particles better in flexibility. A possible reason is as follows.

Fatty acid ethanolamides tend to be highly miscible with cellulose acylates, which can be used as raw materials in the production of cellulosic particles. Using such an ethanolamide as a fatty acid derivative (A), therefore, may encourage the formation of a sea-island structure composed of a sea portion formed by the first component and island portions formed by the second component inside the cellulosic particles, and the region of the island portions, formed by the second component, may tend to grow to a larger size accordingly.

For this reason, presumably, the cellulosic particles may tend to be better in flexibility.

Specifically, the fatty acid ethanolamide may be a compound represented by formula (A-1) below.

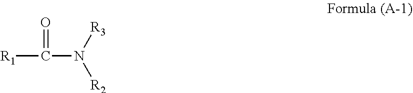

In formula (A-1), R₁ is a residue derived from the fatty acid by removing the carboxy group therefrom; it may be a C10 to C25 saturated aliphatic group, preferably a C15 to C20 saturated aliphatic group.

R₂ and R₃, furthermore, denote residues derived by removing one amino group from ethanolamines. R₂ and R₃ may each be a hydrogen atom or a hydrocarbon group having a hydroxy group. The number of carbon atoms in the hydrocarbon group having a hydroxy group may be one or more and ten or fewer, preferably two or more and five or fewer. R₂ and R₃ may be identical to each other or may be different from each other.

### Aromatic Compound (B)

The aromatic compound having a long-chain aliphatic group and at least one of a phenolic hydroxyl group or a monoglycidyl ether group directly bound to an aromatic group (B) (hereinafter also referred to simply as “aromatic compound (B)”) will now be described.

The long-chain aliphatic group may be a C8 to C20 (or C10 to C18) aliphatic group.

That is, the aromatic compound (B) may be an aromatic compound having a C8 to C20 (or C10 to C18) aliphatic group and at least one of a phenolic hydroxyl group or a monoglycidyl ether group directly bound to an aromatic group (B0) (hereinafter also referred to simply as “aromatic compound (B0)”).

Examples of long-chain aliphatic groups include C8 to C20 (e.g., C10 to C20) saturated aliphatic groups (alkyl groups) and unsaturated aliphatic groups (alkenyl or alkynyl groups). The aliphatic group may be any of linear-chain, branched, or cyclic, but preferably is linear-chain or branched, more preferably linear-chain.

Using an aromatic compound (B0) as an aromatic compound (B) may tend to make the cellulosic particles better in flexibility. A possible reason is as follows.

Aromatic compounds (B0) tend to be highly miscible with cellulose acylates, which can be used as raw materials in the production of cellulosic particles. Using such an aromatic compound as an aromatic compound (B), therefore, may encourage the formation of a sea-island structure composed of a sea portion formed by the first component and island portions formed by the second component inside the cellulosic particles, and the region of the island portions, formed by the second component, may tend to grow to a larger size accordingly.

For this reason, presumably, the cellulosic particles may tend to be better in flexibility.

The octanol/water partition coefficient of the aromatic compound (B0) may be 5 or greater and 20 or less, preferably 7 or greater and 18 or less, more preferably 10 or greater and 15 or less.

The procedure for calculating the octanol/water partition coefficient is as previously described herein.

If the O/W coefficient of the aromatic compound (B0) is 5 or greater and 20 or less, the cellulosic particles may tend to be better in flexibility. A possible reason is as follows.

Aromatic compounds (B0) having an O/W coefficient of 5 or greater and 20 or less tend to be highly miscible with cellulose acylates, which can be used as raw materials in the production of cellulosic particles. Using such an aromatic compound as an aromatic compound (B), therefore, may encourage the formation of a sea-island structure composed of a sea portion formed by the first component and island portions formed by the second component inside the cellulosic particles, and the region of the island portions, formed by the second component, may tend to grow to a larger size accordingly.

For this reason, presumably, the cellulosic particles may tend to be better in flexibility.

Examples of aromatic compounds (B) include compounds derived by substituting a monocycle, a fused system (polycyclic as a result of having two or more aromatic rings), a ring assembly (polycyclic as a result of aromatic rings joined together by a carbon-carbon bond), or a heterocycle (e.g., a heterocyclic monocycle, a fused system including a heterocycle, or a ring assembly including a heterocycle) with a long-chain aliphatic group and a phenolic hydroxyl group.

Specific examples of aromatic compounds (B) include cardanol compounds, phenalkamine compounds, phenolic resins, phenol-novolac epoxy resins, phenol-resol epoxy resins, phenol-modified palm oils, phenol-modified soybean oils, and phenol-modified linseed oils.

In order for the cellulosic particles to be superior in biodegradability and flexibility, the aromatic compound (B) may be a cardanol compound (D1).

“Cardanol compound (D1)” refers to a constituent contained in a compound of natural origin made from cashews (e.g., compounds represented by structural formulae (d-1) to (d-4) below) or a derivative of such a constituent.

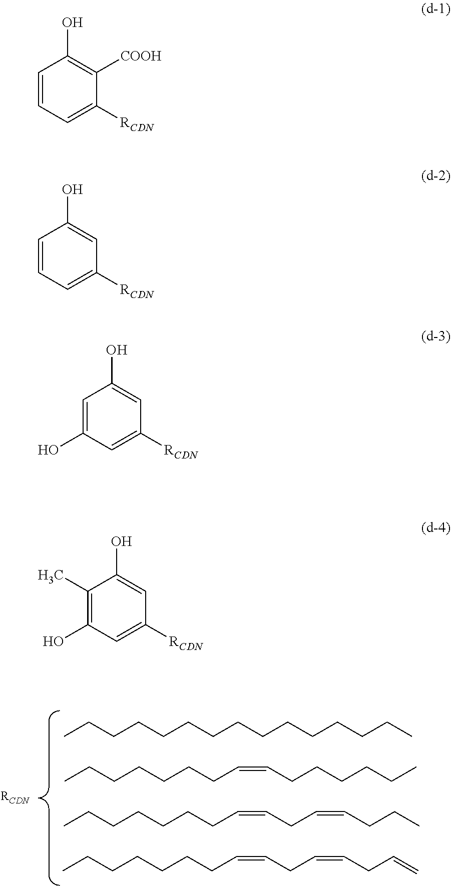

The cardanol compound (D1) may be a mixture of compounds of natural origin made form cashews (hereinafter also referred to as “cashew-derived mixture.”).

The cardanol compound (D1) may be a derivative of a cashew-derived mixture. Examples of derivatives of a cashew-derived mixture include the following mixtures and simple substances.
A composition-adjusted mixture, derived by adjusting the proportions of constituents in a cashew-derived mixture
An isolated simple substance, obtained by isolating a specific constituent from a cashew-derived mixture
A mixture containing a modified form of constituent(s) of a cashew-derived mixture
A mixture containing polymer(s) of constituent(s) of a cashew-derived mixture
A mixture containing polymer(s) of a modified form of constituent(s) of a cashew-derived mixture
A mixture containing a modified form of constituent(s) of a composition-adjusted mixture as described above
A mixture containing polymer(s) of constituent(s) of a composition-adjusted mixture as described above
A mixture containing polymer(s) of a modified form of constituent(s) of a composition-adjusted mixture as described above
A modified form of an isolated simple substance as described above
A polymer of an isolated simple substance as described above
A polymer of a modified form of an isolated simple substance as described above

It should be noted that simple substances in this context include multimers, such as dimers and trimers.

For an improved rate of biodegradation of the cellulosic particles, the cardanol compound (D1) may be at least one compound selected from the group consisting of a compound represented by general formula (CDN1) and a polymer of compounds represented by general formula (CDN1).

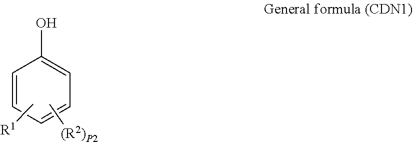

In general formula (CDN1), R¹ represents a substituted or unsubstituted alkyl group or a substituted or unsubstituted unsaturated aliphatic group having double bond(s). R² represents a hydroxy group, a carboxy group, a substituted or unsubstituted alkyl group, or a substituted or unsubstituted unsaturated aliphatic group having double bond(s). P2 represents an integer of 0 to 4. If P2 is 2 or greater, the multiple Res may be identical groups or different groups.

In general formula (CDN1), a substituted or unsubstituted alkyl group represented by R¹ may be a C3 to C30 alkyl group, preferably a C5 to C25 alkyl group, more preferably a C8 to C20 alkyl group.

Examples of substituents include the hydroxy group; substituents containing an ether linkage, such as the epoxy and methoxy groups; and substituents containing an ester linkage, such as the acetyl and propionyl groups.

Examples of substituted or unsubstituted alkyl groups include the pentadecan-1-yl, heptan-1-yl, octan-1-yl, nonan-1-yl, decan-1-yl, undecan-1-yl, dodecan-1-yl, and tetradecan-1-yl groups.

In general formula (CDN1), a substituted or unsubstituted unsaturated aliphatic group having double bond(s) represented by R¹ may be a C3 to C30 unsaturated aliphatic group, preferably a C5 to C25 unsaturated aliphatic group, more preferably a C8 to C20 unsaturated aliphatic group.

The number of double bonds in the unsaturated aliphatic group may be one or more and three or fewer.

Examples of substituents are the same as those listed above as examples of substituents for an alkyl group.

Examples of substituted or unsubstituted unsaturated aliphatic groups having double bond(s) include the pentadeca-8-en-1-yl, pentadeca-8,11-dien-1-yl, pentadeca-8,11,14-trien-1-yl, pentadeca-7-en-1-yl, pentadeca-7,10-dien-1-yl, and pentadeca-7,10,14-trien-1-yl groups.

In general formula (CDN1), R¹ may be a pentadeca-8-en-1-yl, pentadeca-8,11-dien-1-yl, pentadeca-8,11,14-trien-1-yl, pentadeca-7-en-1-yl, pentadeca-7,10-dien-1-yl, or pentadeca-7,10,14-trien-1-yl group.

In general formula (CDN1), examples of substituted or unsubstituted alkyl groups and substituted or unsubstituted unsaturated aliphatic groups having double bond(s) represented by R² are the same as those listed above as examples of substituted or unsubstituted alkyl groups and substituted or unsubstituted unsaturated aliphatic groups having double bond(s) represented by R¹.

The compound represented by general formula (CDN1) may be further modified. For example, it may be epoxidized; specifically, it may be a compound having a structure in which the hydroxy group in the compound represented by general formula (CDN1) has been replaced with group (EP) below, or a compound represented by general formula (CDN1-e) below.

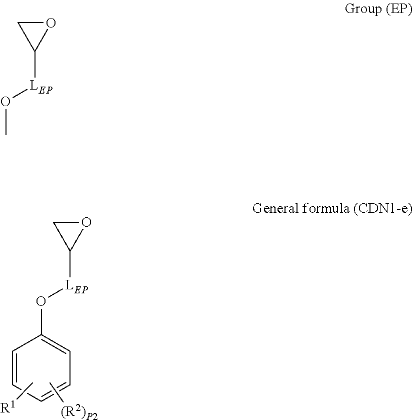

In group (EP) and general formula (CDN1-e), LEP represents a single bond or a divalent linking group. In general formula (CDN1-e), R¹, R², and P2 are synonymous with R¹, R², and P2, respectively, in general formula (CDN1).

In group (EP) and general formula (CDN1-e), examples of divalent linking groups represented by LEP include a substituted or unsubstituted alkylene group (e.g., a C1 to C4 alkylene group, preferably the C1 alkylene group) and the —CH₂CH₂OCH₂CH₂— group.

Examples of substituents are the same as those listed as examples of substituents for R¹ in general formula (CDN1).

LEP may be a methylene group.

The “polymer of compounds represented by general formula (CDN1)” refers to a polymer formed by at least two, or two or more, compounds represented by general formula (CDN1) polymerized together, with or without a linking group therebetween.

An example of a polymer of compounds represented by general formula (CDN1) is a compound represented by general formula (CDN2) below.

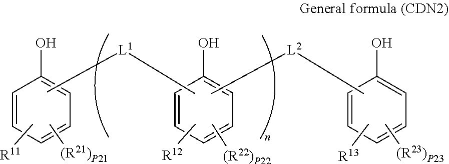

In general formula (CDN2), R¹¹, R¹², and R¹³ each independently represent a substituted or unsubstituted alkyl group or a substituted or unsubstituted unsaturated aliphatic group having double bond(s). R²¹, R²², and R²³ each independently represent a hydroxy group, a carboxy group, a substituted or unsubstituted alkyl group, or a substituted or unsubstituted unsaturated aliphatic group having double bond(s). P21 and P23 each independently represent an integer of 0 to 3, and P22 represents an integer of 0 to 2. L¹ and L² each independently represent a divalent linking group. n represents an integer of 0 to 10. If P21 is 2 or greater, the multiple R²¹s may be identical groups or different groups, if P22 is 2 or greater, the multiple R²²s may be identical groups or different groups, and if P23 is 2 or greater, the multiple R²³s may be identical groups or different groups. If n is 2 or greater, the multiple R¹²s may be identical groups or different groups, the multiple R²²s may be identical groups or different groups, and the multiple L¹s may be identical groups or different groups, and if n is 2 or greater, the multiple P22s may be the same number or different numbers.

In general formula (CDN2), examples of substituted or unsubstituted alkyl groups and substituted or unsubstituted unsaturated aliphatic groups having double bond(s) represented by R¹¹, R¹², R¹³, R²¹, R²², and R²³ are the same as those listed as examples of R¹s in general formula (CDN1).

In general formula (CDN2), examples of divalent linking groups represented by L¹ and L² include a substituted or unsubstituted alkylene group (e.g., a C2 to C30 alkylene group, preferably a C5 to C20 alkylene group).

Examples of substituents are the same as those listed as examples of substituents for R¹ in general formula (CDN1).

In general formula (CDN2), n may be 1 or greater and 10 or less, preferably 1 or greater and 5 or less.

The compound represented by general formula (CDN2) may be further modified. For example, it may be epoxidized; specifically, it may be a compound having a structure in which the hydroxy groups in the compound represented by general formula (CDN2) have been replaced with group (EP), or a compound represented by general formula (CDN2-e) below.

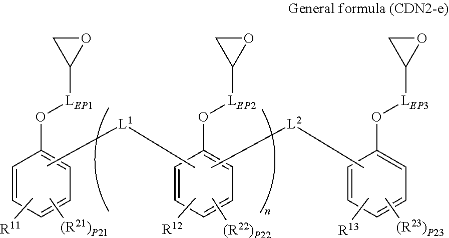

In general formula (CDN2-e), R¹¹, R¹², R¹³, R²¹, R²², R²³, P²¹, P²², P²³, L¹, L², and n are synonymous with R¹¹, R¹², R¹³, R²¹, R²², R²³, F²¹, P²², P²³, L¹, L², and n, respectively, in general formula (CDN2).

In general formula (CDN2-e), L_{EP1}, L_{EP2}, and L_{EP3} each independently represent a single bond or a divalent linking group. If n is 2 or greater, the multiple L_{EP2}s may be identical groups or different groups.

In general formula (CDN2-e), examples of divalent linking groups represented by L_{EP1}, L_{EP2}, and L_{EP3} are the same as those listed as examples of divalent linking groups represented by LEP in general formula (CDN1-e).

The polymer of compounds represented by general formula (CDN1) may be, for example, a polymer formed by at least three, or three or more, compounds represented by general formula (CDN1) three-dimensionally polymerized together by crosslinking, with or without a linking group therebetween. An example of a polymer formed by compounds represented by general formula (CDN1) three-dimensionally polymerized together by crosslinking is a compound represented by the following structural formula.

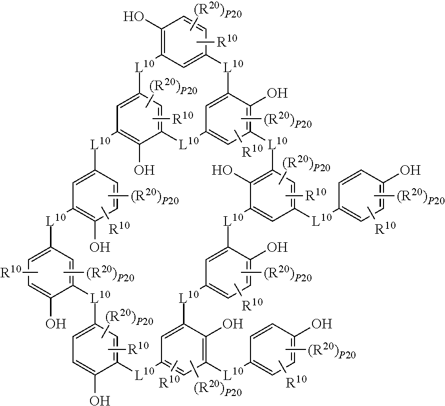

In this structural formula, R¹⁰, R²⁰, and P20 are synonymous with R¹, R², and P2, respectively, in general formula (CDN1). L¹⁰ represents a single bond or a divalent linking group. The multiple R¹⁰s may be identical groups or different groups, multiple R²⁰s may be identical groups or different groups, and the multiple L¹⁰s may be identical groups or different groups. The multiple P20s may be the same number or different numbers.

In this structural formula, an example of a divalent linking group represented by L¹⁰ is a substituted or unsubstituted alkylene group (e.g., a C2 to C30 alkylene group, preferably a C5 to C20 alkylene group).

Examples of substituents are the same as those listed as examples of substituents for R¹ in general formula (CDN1).

The compound represented by the above structural formula may be further modified; for example, it may be epoxidized. Specifically, it may be a compound having a structure in which the hydroxy groups in the compound represented by the above structural formula have been replaced with group (EP); an example is a compound represented by the following structural formula, that is, a polymer formed by compounds represented by general formula (CDN1-e) three-dimensionally polymerized together by crosslinking.

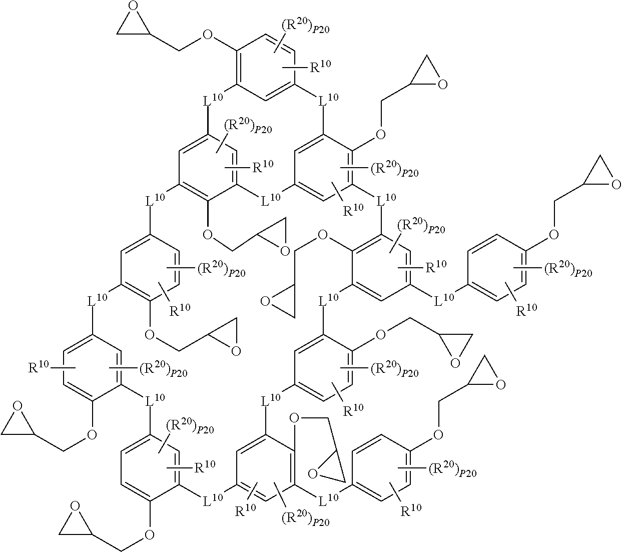

In this structural formula, R¹⁰, R²⁰, and P20 are synonymous with R¹, R², and P2, respectively, in general formula (CDN1-e). L¹⁰ represents a single bond or a divalent linking group. The multiple R¹⁰s may be identical groups or different groups, multiple R²⁰s may be identical groups or different groups, and the multiple L¹⁰s may be identical groups or different groups. The multiple P20s may be the same number or different numbers.

In this structural formula, an example of a divalent linking group represented by L¹⁰ is a substituted or unsubstituted alkylene group (e.g., a C2 to C30 alkylene group, preferably a C5 to C20 alkylene group).

Examples of substituents are the same as those listed as examples of substituents for R¹ in general formula (CDN1).

The cardanol compound (D1) may be a commercially available one. Examples of commercially available cardanol compounds include Cardolite's NX-2024, Ultra LITE 2023, NX-2026, GX-2503, NC-510 LITE 2020, NX-9001, NX-9004, NX-9007, NX-9008, NX-9201, and NX-9203 and Tohoku Chemical Industries' LB-7000, LB-7250, and CD-5L. Examples of commercially available epoxy-containing cardanol compounds include Cardolite's NC-513, NC-514S, NC-547, LITE513E, and Ultra LTE 513.

For an improved rate of biodegradation of the cellulosic particles, the hydroxyl number of the cardanol compound (D1) may be 100 mg KOH/g or more, preferably 120 mg KOH/g or more, more preferably 150 mg KOH/g or more. The measurement of the hydroxyl number of a cardanol compound (D1) is done according to Method A in ISO 14900.

If the cardanol compound (D1) is an epoxy-containing cardanol compound (D1), its epoxy equivalent may be 300 or more and 500 or less, preferably 350 or more and 480 or less, more preferably 400 or more and 470 or less for improved transparency of the cellulosic particles. The measurement of the epoxy equivalent of an epoxy-containing cardanol compound (D1) is done according to ISO 3001.

For improved heat resistance and flexibility of the cellulosic particles, the weight-average molecular weight of the cardanol compound (D1) may be 250 or more and 1000 or less, preferably 280 or more and 800 or less, more preferably 300 or more and 500 or less.

The weight-average molecular weight of a cardanol compound (D1) is measured as a polystyrene-equivalent value on a gel permeation chromatograph (GPC; Tosoh, HLC-8320GPC; column, TSKgel α-M) using tetrahydrofuran eluent.

One cardanol compound (D1) may be used alone, or two or more may be used in combination.

### (Meth)Acrylic Compound (C)

A (meth)acrylic compound (C) is a polymer formed by a unit monomer that is at least one selected from the group consisting of (meth)acrylic acid and a (meth)acrylic acid derivative.

In this context, “(meth)acrylic” means acrylic or methacrylic.

A (meth)acrylic derivative is a compound derived by allowing the carboxy group of (meth)acrylic acid with another functional group; examples include (meth)acrylamides and (meth)acrylates.

The (meth)acrylic acid derivative may be a (meth)acrylate.

A (meth)acrylate is a compound derived by esterifying the carboxy group of (meth)acrylic acid with a compound having a hydroxy group and has at least one ester group.

Examples of (meth)acrylates include C1 to C30 (or C1 to C20) alkyl or hydroxyalkyl (meth)acrylates and glycerylamidoethyl methacrylate.

The (meth)acrylic compound (C) may have a crosslink structure.

The (meth)acrylic compound (C) may include, as its repeat unit, an extra monomer that is neither (meth)acrylic acid nor a (meth)acrylic acid derivative.

An example of an extra monomer is vinylpyrrolidone.

Examples of (meth)acrylic compounds (C) include the compounds identified by the INCI names of “ACRYLATES/C10-30 ALKYL ACRYLATE CROSSPOLYMER,” “ACRYLATES/ETHYLHEXYL ACRYLATE/DIMETHICONE METHACRYLATE COPOLYMER,” “ACRYLIC ACID/VP CROSSPOLYMER,” “ACRYLATES/HYDROXYESTERS ACRYLATES COPOLYMER,” “ACRYLATES/C10-30 ALKYL ACRYLATE CROSSPOLYMER,” “ACRYLATES/C1-2 SUCCINATES/HYDROXYACRYLATES COPOLYMER,” and “POLY C10-30 ALKYL ACRYLATE.”

### Total Amount of the Second Component

The total amount of the second component may be 5% by mass or more and 30% by mass or less, preferably 10% by mass or more and 25% by mass or less, more preferably 15% by mass or more and 20% by mass or less of the total amount of the first and second components.

Making the amount of the second component 5% by mass or more and 30% by mass or less of the total amount of the first and second components may tend to make the cellulosic particles better in biodegradability and flexibility. A possible reason is as follows.

Making the amount of the second component 5% by mass or more of the total amount of the first and second components may encourage the growth of the region of the island portions, formed by the second component, to a large size; the flexibility of the cellulosic particles, therefore, may be improved.

Making the amount of the second component 30% by mass or less of the total amount of the first and second components may help ensure that the amount of the second component in the cellulosic particles is not too large. This may help limit the associated decrease in the biodegradability of the cellulosic particles.

For this reason, presumably, ensuring that the amount of the first component falls within the above range may tend to make the cellulosic particles better in biodegradability and flexibility.

### Cellulosic Particles Having a Coating Layer

The cellulosic particles according to this exemplary embodiment may be cellulosic particles each including a core particle containing the first and second components and a coating layer covering the core particle and containing at least one selected from the group consisting of a polyamine compound, a wax, a linear-chain saturated fatty acid, a hydroxy fatty acid, and an amino acid compound (hereinafter also referred to as “cellulosic particles having a coating layer”).

Configuring the cellulosic particles according to this exemplary embodiment in this way may tend to make the cellulosic particles better in flexibility. A possible reason is as follows.

A polyamine compound or linear-chain saturated fatty acid tends to form a structure in which its relatively long linear chain seems like extending toward the outside of the particles because its amino group or carboxylic acid structure has an ionic affinity for hydroxyl groups in the cellulose. A hydroxy fatty acid, whose hydroxyl group forms hydrogen bonds with hydroxyl groups in the cellulose, stretches its relatively long linear chain over the surface of the particles at a certain angle starting from the hydroxyl groups; the linear chains become entangled with each other, often forming a spongelike structure. With such an ability to form a coating layer in a higher-order structure, these compounds may absorb external forces applied to the particles by deforming this higher-order structure and, as a result, produce superior flexibility. A wax and an amino acid compound self-aggregate on the cellulose surface and both tend to be shaped like flat islands with adequate spaces therebetween when covering the particle surface. By virtue of being present in an islet shape, presumably, these compounds may be highly effective in absorbing external forces and produce superior flexibility, even in small amounts.

For this reason, presumably, the cellulosic particles may tend to be better in flexibility.

### Core Particle

The core particle contains the first and second components.

The first and second components contained in the core particle have the same definition as the first and second components, respectively, previously described herein; possible and preferred ranges of parameters are also the same as in the foregoing.

### Coating Layer

The coating layer contains at least one selected from the group consisting of a polyamine compound, a wax, a linear-chain saturated fatty acid, a hydroxy fatty acid, and an amino acid compound.

Polyamine Compound

“Polyamine compound” is a generic term for aliphatic hydrocarbons having two or more primary amino groups.

Examples of polyamine compounds include a polyalkyleneimine, polyallylamine, polyvinylamine, and polylysine.

For improved biodegradability, the polyalkyleneimine may be a polyalkyleneimine including a repeat unit having a C1 to C6 (preferably C1 to C4, more preferably C1 or C2) alkylene group, preferably polyethyleneimine.

Examples of polyallylamines include homopolymers or copolymers of allylamine, allylamine amidosulfate, diallylamine, dimethylallylamine, etc.

Examples of polyvinylamines include products of alkali hydrolysis of poly(N-vinylformamide); a specific example is Mitsubishi Chemical's “PVAM-0595B.”

The polylysine may be an extract from a natural source, may be a substance produced by a transformed microorganism, or may be a product of chemical synthesis.

The polyamine compound may be at least one selected from the group consisting of polyethyleneimine and polylysine.

Using at least one selected from the group consisting of polyethyleneimine and polylysine as polyamine compound(s) may tend to make the cellulosic particles better in flexibility. A possible reason is as follows.

Among polyamine compounds, polyethyleneimine and polylysine are particularly highly cationic and have a higher affinity than others for cellulosic hydroxyl groups. They, therefore, are adsorbed firmly onto the cellulosic particles and do not easily detach during the production or use of the particles; this may render the cellulosic particles better in flexibility.

For this reason, presumably, the cellulosic particles may tend to be better in flexibility.

The polyamine compound content may be 0.2% by mass or more and 2% by mass or less of the cellulosic particles as a whole.

Wax

Examples of waxes include fatty acid-containing vegetable oils, hydrocarbon waxes, and diesters.

Examples of fatty acid-containing vegetable oils include castor oil, paulownia oil, linseed oil, shortening, corn oil, soybean oil, sesame oil, rapeseed oil, sunflower oil, rice bran oil, camellia oil, coconut oil, palm oil, walnut oil, olive oil, peanut oil, almond oil, jojoba oil, cocoa butter, shea butter, neem oil, safflower oil, Japan wax, candelilla wax, rice bran wax, carnauba wax, and Rosa damascena flower wax.

Examples of hydrocarbon waxes include petroleum waxes (paraffin wax, microcrystalline wax, petrolatum wax, etc.) and synthetic hydrocarbon waxes (polyethylene wax, polypropylene wax, polybutene wax, Fischer-Tropsch wax, etc.).

Examples of diesters include diesters of dibasic acids, such as malic acid, glutaric acid, adipic acid, azelaic acid, sebacic acid, and dodecanedioic acid, and C10 to C25 alcohols.

The wax may be carnauba wax.

Using carnauba wax as a wax may tend to make the cellulosic particles better in flexibility. A possible reason is as follows.

Carnauba wax contains fatty acids as its constituents. The terminal carboxylic acid of the fatty acids is adsorbed firmly onto the particle surface by virtue of its high affinity for hydroxyl groups in the cellulose and, therefore, probably does not easily detach during the production or use of the particles. This may allow the cellulosic particles to produce better flexibility.

For this reason, presumably, the cellulosic particles may tend to be better in flexibility.

The wax content may be 0.1% by mass or more and 2% by mass or less, preferably 0.2% by mass or more and 1% by mass or less, of the cellulosic particles as a whole.

Linear-Chain Saturated Fatty Acid

Linear-chain saturated fatty acids are saturated fatty acids in a linear-chain structure.

In order for the cellulosic particles to be better in flexibility or for the cellulosic particles to be better in biodegradability, the linear-chain saturated fatty acid may be a C14 to C22 linear-chain saturated fatty acid.

Examples of C14 to C22 linear-chain saturated fatty acids include behenic acid, arachidic acid, and palmitic acid.

The linear-chain saturated fatty acid content may be 2% by mass or more and 15% by mass or less, preferably 5% by mass or more and 10% by mass or less, of the cellulosic particles as a whole.

Hydroxy Fatty Acid

Hydroxy fatty acids are fatty acids having a hydroxy group.

An example of a hydroxy fatty acid is a C14 to C20 hydroxy fatty acid.

Examples of C14 to C20 hydroxy fatty acids include hydroxystearic acid, hydroxypalmitic acid, and hydroxymyristic acid.

The hydroxy fatty acid content may be 1% by mass or more and 10% by mass or less, preferably 3% by mass or more and 10% by mass or less, of the cellulosic particles as a whole.

Amino Acid Compound

“Amino acid compounds” refers to amino acids and amino acid derivatives.

An amino acid derivative is a compound derived from an amino acid by replacing one or more hydrogen atoms or functional groups therein with another substituent.

The amino acid compound may be an amino acid derivative.

Examples of amino acid derivatives include lauroyl lysine, lauryl arginine, and myristyl leucine.

The amino acid compound content may be 2% by mass or more and 10% by mass or less of the cellulosic particles as a whole.

Layer Structure of the Coating Layer

The coating layer may have a first coating layer covering the core particle and containing at least one selected from the group consisting of a polyamine compound, polyvinyl alcohol, polyvinylpyrrolidone, a linear-chain saturated fatty acid, a hydroxy fatty acid, and an amino acid compound and a second coating layer covering the first coating layer and containing a wax.

In particular, the coating layer may have a first coating layer covering the core particle and containing at least one selected from the group consisting of a polyamine compound, a linear-chain saturated fatty acid, and a hydroxy fatty acid and a second coating layer covering the first coating layer and containing a wax.

The presence of such first and second coating layers in the coating layer may tend to make the cellulosic particles better in flexibility. A possible reason is as follows.

As stated, a polyamine compound, a linear-chain saturated fatty acid, and a hydroxy fatty acid are all adsorbed onto the particle surface by virtue of their affinity for hydroxyl groups in the cellulose, with their relatively long linear-chain structure facing toward the outside or entangled between molecules on the particle surface. If carnauba wax is allowed to act on this system, the carnauba wax self-aggregates and forms an islet structure on the surface of the first layer. As a result of the formation of flexible islands on a cushion formed by the first layer, better flexibility may be produced.

For this reason, presumably, the cellulosic particles may tend to be better in flexibility.

Polyvalent Metal Salt

The second coating layer may contain a polyvalent metal salt.

The presence of a polyvalent metal salt in the second coating layer may tend to make the cellulosic particles better in flexibility. A possible reason is as follows.

The wax contained in the second layer adheres to the layer beneath it only weakly. The resulting coating, therefore, tends to have many defects as a result of the self-aggregation of the wax. A polyvalent metal salt contained in the second coating layer together with the wax spreads nearly uniformly throughout the wax and may provide starting points for the wax to aggregate nearly uniformly and extensively; the formation of coating defects due to the self-aggregation of the wax, therefore, may be limited, and the adhesion of the second coating layer may be improved. An improved adhesion of the second coating layer, furthermore, may tend to improve the flexibility of the cellulosic particles.

For this reason, presumably, the cellulosic particles may tend to be better in flexibility.

Polyvalent metal salts are compounds formed by a divalent or higher-valency metal ion and an anion.

Examples of divalent or higher-valency metal ions as a component of a polyvalent metal salt include the ions of calcium, magnesium, copper, nickel, zinc, barium, aluminum, titanium, strontium, chromium, cobalt, iron, etc.

Examples of anions as a component of a polyvalent metal salt include inorganic or organic ions. Examples of inorganic ions include the chloride, bromide, iodide, nitrate, sulfate, and hydroxide ions. Examples of organic ions include organic acid ions, such as the carboxylate ion.

Examples of polyvalent metal salts include aluminum sulfate, polyaluminum chloride, iron chloride, and calcium hydroxide.

The polyvalent metal salt content relative to the wax content may be 0.1% by mass or more and 10% by mass or less, preferably 0.2% by mass or more and 5% by mass or less, more preferably 0.3% by mass or more and 1% by mass or less.

Amounts of Constituents in the First and Second Coating Layers

The total amount of the polyamine compound, polyvinyl alcohol, polyvinylpyrrolidone, linear-chain saturated fatty acid, hydroxy fatty acid, and/or amino acid compound relative to the entire first coating layer may be 90% by mass or more and 100% by mass or less, preferably 95% by mass or more and 100% by mass or less.

The total amount of the wax and polyvalent metal salt relative to the entire second coating layer may be 90% by mass or more and 100% by mass or less, preferably 95% by mass or more and 100% by mass or less.

### External Additive(s)

The cellulosic particles according to this exemplary embodiment may have at least one external additive selected from the group consisting of silicon-containing compound particles, metallic soap particles, amino acid compound particles, fatty acid ester particles, metal oxide particles, and hydroxy fatty acid particles.

In particular, the cellulosic particles according to this exemplary embodiment may have at least one external additive selected from the group consisting of silicon-containing compound particles and metallic soap particles.

The presence of such external additive(s) may tend to make the cellulosic particles according to this exemplary embodiment better in flexibility. A possible reason is as follows.

Silicon-containing compound particles adhere to the cellulosic particles by electrostatic adhesion. The adhering silicon-containing compound particles are softer than the cellulosic particles; when an external force acts on the particles, therefore, the silicon-containing particles deform first, producing flexibility. If the cellulosic particles were known ones, they would not deform because of their hardness, and their flexibility would be limited to the degree permitted by the particle diameter of the silicon-containing particles; the cellulosic particles according to this exemplary embodiment, however, may have their own flexibility and, therefore, may deform after the silicon compound deforms to some extent. By virtue of this, the cellulosic particles may produce superior flexibility.

Metallic soap particles, which adhere to the cellulosic particles by partial fusion, are slightly softer than the cellulosic particles, and the reason why they may give the cellulosic particles superior flexibility is similar to that with silicon-containing particles.

For this reason, presumably, the cellulosic particles may tend to be better in flexibility.

“Silicon-containing compound particles” refers to particles containing silicon.

The silicon-containing compound particles may be particles of silicon or may be particles containing silicon and other element(s).

The silicon-containing compound particles may be silica particles.

The silica particles can be any silica-based, or SiO₂-based, particles, whether crystalline or amorphous. The silica particles, furthermore, may be particles produced from a raw-material silicon compound, such as waterglass or an alkoxysilane, or may be particles obtained by crushing quartz.

Using silica particles as silicon-containing compound particles may tend to make the cellulosic particles better in flexibility. A possible reason is as follows.

Silica particles, among other kinds of silicon-containing compound particles, adhere particularly firmly to the cellulosic particles by electrostatic adhesion. Events like a slide and subsequent detachment of the silica particles after an external force acts on the cellulosic particles, therefore, are rare, and this may allow the cellulosic particles to produce better flexibility.

For this reason, presumably, the cellulosic particles may tend to be better in flexibility.

Metallic soap particles are metallic soap-based particles.

In this context, “metallic soap-based particles” refers to particles containing 90% by mass or more metallic soap in relation to the particles themselves.

A metallic soap is a metallic salt of a fatty acid, formed by a fatty acid and a metal bound together.

An example of a metallic salt of a fatty acid is a metallic salt of a C10 to C25 (preferably C12 to C22) fatty acid. Examples of metallic salts of C10 to C25 fatty acids include metallic salts of stearic acid, palmitic acid, lauric acid, oleic acid, linoleic acid, and ricinoleic acid.

An example of a metal in a metallic salt of a fatty acid is a divalent metal.

Examples of metals in metallic salts of fatty acids include magnesium, calcium, aluminum, barium, and zinc.

Fatty acid ester particles are particles including fatty acid ester particles as a base component.

In this context, “particles including fatty acid ester particles as a base component” refers to particles including 90% by mass or more fatty acid ester particles in relation to the particles themselves.

An example of a fatty acid ester is the product of esterification between a C10 to C25 saturated fatty acid and a C10 to C25 alcohol.

Examples of fatty acid esters include stearyl stearate, stearyl laurate, and stearyl palmitate.

Metal oxide particles are metal oxide-based particles.

In this context, “metal oxide-based particles” refers to particles containing 90% by mass or more metal oxide in relation to the particles themselves.

The metal oxide can be an oxide of a metal other than silicon.

Examples of metal oxides include zinc oxide, magnesium oxide, iron oxide, aluminum oxide, and calcium oxide.

The amount of the external additive may be 0.01% by mass or more and 2% by mass or less of the mass of the cellulosic particles (cellulosic particles to which the external additive has yet to be added) as a whole.

### Volume-Average Particle Diameter and Upper Geometric Standard Deviation by Number GSDv

The volume-average particle diameter of the cellulosic particles according to this exemplary embodiment may be 3 μm or more and less than 10 μm, preferably 4 μm or more and 9 μm or less, more preferably 5 μm or more and 8 μm or less.

Making the volume-average particle diameter of the cellulosic particles according to this exemplary embodiment 3 μm or more and less than 10 μm may tend to make the cellulosic particles better in flexibility. A possible reason is as follows.

If the volume-average particle diameter of the cellulosic particles is 3 μm or more, an increased physical limit to which the particles can deform in response to an external force may tend to improve the production of flexibility by the cellulosic particles. The cellulosic particles according to this exemplary embodiment may be superior in surface flexibility in particular, so if the volume-average particle diameter of the cellulosic particles is 10 μm or less, a reduced relative volume of the core compared with the surface may tend to improve the production of flexibility by the cellulosic particles. Overall, if the volume-average particle diameter of the cellulosic particles is 3 μm or more and 10 μm or less, the particles may tend to produce superior flexibility because in that case a sufficiently large amount of deformation may be combined with a large relative volume of the surface.

For this reason, presumably, the cellulosic particles may tend to be better in flexibility.

The upper geometric standard deviation by number GSDv of the cellulosic particles according to this exemplary embodiment may be 1.0 or greater and 1.7 or less, preferably 1.0 or greater and 1.5 or less, more preferably 1.0 or greater and 1.3 or less.

Making the upper geometric standard deviation by number GSDv of the cellulosic particles according to this exemplary embodiment 1.0 or greater and 1.7 or less may tend to make the cellulosic particles better in flexibility. A possible reason is as follows.

If the GSDv is 1.7 or less, reduced numbers and percentages of fine particles and coarse particles may often lead to a greater deformation of the particles and may ensure the presence of particles with a high percentage volume of the surface; the flexibility advantage, therefore, may tend to be more significant.

For this reason, presumably, the cellulosic particles may tend to be better in flexibility.

The volume-average particle diameter and the upper geometric standard deviation GSDp of the cellulosic particles are measured as follows.

Particle diameters are measured using the LS particle size distribution analyzer “Beckman Coulter LS13 320 (Beckman Coulter),” and the cumulative distribution of particle diameters is plotted as a function of volume starting from the smallest diameter; then the particle diameter at which the cumulative percentage is 50% is determined as the volume-average particle diameter.

Separately, the cumulative distribution of particle diameters is plotted as a function of volume starting from the smallest diameter, and the particle diameters at which the cumulative percentage is 50% and 84% are defined as the number-average particle diameter, D50v, and particle diameter D84v by number, respectively. The upper geometric standard deviation by number GSDv is calculated according to the equation GSDv=(D84v/D50v)^{1/2}.

### Sphericity

The sphericity of the cellulosic particles according to this exemplary embodiment may be 0.90 or greater, preferably 0.95 or greater, more preferably 0.97 or greater.

Making the sphericity of the cellulosic particles according to this exemplary embodiment 0.90 or greater may tend to make the cellulosic particles better in flexibility. A possible reason is as follows.

Cellulose is a crystalline polymer, and if particles of cellulose have protrusions on their surface, the cellulose easily crystallizes there. The higher the degree of crystallinity is, furthermore, the harder the cellulosic particles are. Making the sphericity 0.90 or greater may ensure that the percentage area of protrusions is small, and this may allow the cellulosic particles to produce superior flexibility.

For this reason, presumably, the cellulosic particles may tend to be better in flexibility.

The sphericity is given by (circumference of the equivalent circle)/(circumference) [(circumference of a circle having the same projected area as the particle's image)/(circumference of the particle's projected image)]. Specifically, the sphericity is a value measured by the following method.

First, a portion of the cellulosic particles of interest is collected by aspiration in such a manner that it will form a flat stream, and this flat stream is photographed with a flash to capture the figures of the particles in a still image; then the particle images obtained are analyzed using a flow particle-image analyzer (Sysmex Corp. FPIA-3000) to give the sphericity. The number of particles sampled in the determination of the sphericity is 3500.

If the cellulosic particles have an external additive, the cellulosic particles of interest are dispersed in water containing a surfactant and then sonicated to eliminate the external additive, and the sonicated particles are subjected to the measurement.

### Surface Smoothness

The surface smoothness of the cellulosic particles according to this exemplary embodiment may be 80% or higher, preferably 82% or higher and 99% or lower, more preferably 84% or higher and 98% or lower.

Making the surface smoothness of the cellulosic particles according to this exemplary embodiment 80% or higher may tend to make the cellulosic particles better in biodegradability and flexibility. A possible reason is as follows.

Making the surface smoothness 80% or higher means reducing the number of protrusions on the surface. The force of repulsion of the particles against external stress at their portions derived from surface protrusions may decrease accordingly, and this may encourage the improvement of flexibility. In that case, furthermore, the contact between the cellulosic particles and microorganisms may be close to uniformity, and the biodegradation process may tend to proceed nearly uniformly on the surface of the cellulosic particles; biodegradability, therefore, may tend to be improved.

For this reason, presumably, the cellulosic particles may tend to be better in biodegradability and flexibility.

The surface smoothness is measured through a procedure as described below.

An SEM image (magnification, 5,000 times) of the cellulosic particles, taken with a scanning electron microscope (SEM), is observed, and the smoothness M of the individual cellulosic particles is calculated according to the equation below. Then the arithmetic mean smoothness M of any ten or more cellulosic particles is reported as the surface smoothness. The closer the smoothness M is to 1, the closer the surface of the cellulosic particles is to smoothness.

M=(1−(S3)/(S2))×100

In this equation, S2 denotes the area of the cellulosic particle in the image (projected area), and S3 denotes, when the cellulosic particle in the image is superimposed on a circle having a projected area equal to S2, the sum of “the area outside the outline of the circle having a projected area equal to S2 and inside the outline of the cellulosic particle in the image” and “the area inside the outline of the circle having a projected area equal to S2 and outside the outline of the cellulosic particle in the image.”

The method for superimposing the cellulosic particle in the image on a circle having a projected area equal to S2 is as follows.

The cellulosic particle in the image is superimposed on the circle having a projected area equal to S2 so that the area of overlap between the two images (the area inside the outline of the circle having a projected area equal to S2 and inside the outline of the cellulosic particle in the image) will be maximized.

### Method for Producing the Cellulosic Particles

A method for producing the cellulosic particles may include a step of producing a particle precursor containing a cellulose acylate and the second component (particle precursor production step) and a step of saponifying the cellulose acylate contained in the particle precursor (saponification step).

### Particle Precursor Production Step

A particle precursor containing a cellulose acylate and the second component (at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C)) is produced by any of methods (1) to (5) below.

(1) Kneading and milling, in which the ingredients are kneaded together, and the resulting mixture is milled and classified to give grains

(2) A dry process, in which the shape of the grains obtained by kneading and milling is changed with the help of a mechanical impact force or thermal energy

(3) Aggregation and coalescence, in which particle dispersions of the ingredients are mixed together, and the particles in the mixed dispersion are caused to aggregate and fused together under heat to give grains

(4) Dissolution and suspension, in which a solution of the ingredients in an organic solvent is suspended in an aqueous medium to form grains containing the ingredients

(5) Kneading and dissolution, in which the ingredients and a binding material are kneaded together, the resulting mixture is pelletized by extrusion, and the resulting pellets are stirred in a solvent for the binder to form grains

The “ingredients” mentioned in (1) to (5) above represent ingredients including a cellulose acylate and at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C).

In this context, a cellulose acylate is a cellulose derivative in which at least one of the hydroxy groups in cellulose has been replaced with an aliphatic acyl group (acylated). Specifically, a cellulose acylate is a cellulose derivative in which at least one of the hydroxy groups in cellulose has been replaced with —CO—R^{AC} (R^{AC} represents an aliphatic hydrocarbon group.).

### Saponification Step

Then the cellulose acylate contained in the particle precursor is saponified.

Through this step, the aliphatic acyl group(s) in the cellulose acylate is hydrolyzed, and the cellulose turns into cellulose.

The saponification step is performed by, for example, adding sodium hydroxide to a dispersion of the particle precursor and stirring the dispersion.

### Coating Layer Formation Step

If cellulosic particles having a coating layer are produced, the production method may include a step of forming the coating layer (coating layer formation step) after the above saponification step.

If the coating layer formation step is performed, the coating layer is formed using the particles obtained through the above saponification step as core particles.

First, an aqueous dispersion in which the core particles are dispersed is prepared. The core particles may be cleaned with acid before the preparation of the aqueous dispersion.

Then the aqueous dispersion in which the core particles are dispersed is mixed with an aqueous solution containing the compound(s) that will form the first coating layer. This causes, for example, hydroxyl groups in the resin contained in the core particles to react, for example with the amine sites or carboxylic acid sites in the surface-treating polymer(s), to form the first coating layer. Then the aqueous dispersion in which the core particles with the first coating layer formed thereon are dispersed is mixed with an emulsion containing the compound(s) that will form the second coating layer. Through this, the second coating layer is formed.

Then the cellulosic particles having coating layers are removed from the mixture. The removal of the cellulosic particles having coating layers is done by, for example, filtering the mixture. The removed cellulosic particles having coating layers may be washed with water. This may help eliminate unreacted residue of the surface-treating polymer(s). Then the cellulosic particles having coating layers are dried, giving cellulosic particles according to this exemplary embodiment.

### Addition Step

External additive(s) may be added to the resulting cellulosic particles.

An example of an addition step is a treatment in which the external additive(s) is added to the cellulosic particles using equipment like a mixing mill, V-blender, Henschel mixer, or Lödige mixer.

Producing the cellulosic particles according to this exemplary embodiment through these steps may make it more certain that cellulosic particles superior in biodegradability and flexibility are obtained. The reason is unclear, but a possible reason is as follows.

The second component tends to be miscible with cellulose acylates to some extent. In the resin particle precursor obtained in the above resin particle precursor production step, therefore, the cellulose acylate and the second component are dissolved in each other to some extent. Once the cellulose acylate turns into cellulose in the subsequent saponification step, it loses miscibility with the second component, and this appears to encourage the formation of a sea-island structure composed of a sea portion formed by the first component and island portions formed by the second component inside the cellulosic particles.

For this reason, presumably, producing the cellulosic particles according to this exemplary embodiment through the above steps may make it more certain that cellulosic particles superior in biodegradability and flexibility are obtained.

### Applications

Applications of the cellulosic particles according to this exemplary embodiment include grains for use as cosmetics, a rolling agent, an abrasive, a scrubbing agent, display spacers, a material for bead molding, light-diffusing particles, a resin-strengthening agent, a refractive index control agent, a biodegradation accelerator, a fertilizer, water-absorbent particles, toner particles, and anti-blocking particles.

An application of the cellulosic particles according to this exemplary embodiment may be cosmetics.

An application of the cellulosic particles according to this exemplary embodiment may be a cosmetic additive in particular.

Potentially superior in flexibility, the cellulosic particles according to this exemplary embodiment, if used as a cosmetic additive, may help the cosmetic product to spread well on the skin when the cosmetic product is put on the skin.

The cellulosic particles according to this exemplary embodiment can be applied as cosmetic additives, for example to base makeup cosmetics (e.g., foundation primer, concealer, foundation, and face powder); makeup cosmetics (e.g., lipstick, lip gloss, lip liner, blush, eye shadow, eyeliner, mascara, eyebrow powder, nail products, and nail care cosmetics); and skincare cosmetics (e.g., face wash, facial cleanser, toner, milky lotion, serum, face packs, face masks, and cosmetics for the care of the eye and mouth areas).

The resin particles according to this exemplary embodiment may be used as a cosmetic additive to makeup cosmetics in particular, because cosmetic additives to makeup cosmetics can need to be flexible and biodegradable.

### EXAMPLES

Examples will now be described, but no aspect of the present disclosure is limited to these examples. In the following description, “parts” and “%” are all by mass unless stated otherwise.

### Preparation of Materials

The following materials are prepared.

### Cellulose Acylates

Cel1: Daicel “L-20”; cellulose acetate; number-average molecular weight, 47000.
Cel2: Daicel “L-50”; cellulose acetate; number-average molecular weight, 58000.
Cel3: Eastman Chemical “CAP482-20”; cellulose acetate propionate; number-average molecular weight, 75000.
Cel4: Eastman Chemical “CAB381-20”; cellulose acetate butyrate; number-average molecular weight, 70000.
Cel5: Eastman Chemical “CA398-6”; cellulose acetate; number-average molecular weight, 35000.
Cel6: Eastman Chemical “CAP482-0.5”; cellulose acetate propionate; number-average molecular weight, 25000.
Cel7: Eastman Chemical “CAP-504-0.2”; cellulose acetate propionate; number-average molecular weight, 15000.

### Second Component

### Fatty Acid Derivatives (A)

Add1: Kawaken Fine Chemicals “AMISOL ODE,” oleic acid diethanolamide
Add2: Kawaken Fine Chemicals “AMISOL CME,” coconut fatty acid monoethanolamide
Add21: Kawaken Fine Chemicals “AMISOL SDHE,” stearic acid diethanolamide
Add22: Kawaken Fine Chemicals “AMISOL PLME-A,” lauric acid monoisopropanolamide

### (Meth)acrylic Compounds (C)

Add3: Sumitomo Seika Chemicals “AQUPEC HV-501ER”; INCI name, “ACRYLATES/C10-30 ALKYL ACRYLATE CROSSPOLYMER”
Add4: Sekisui Kasei “ACP-8C”; INCI name, “ACRYLATES/ETHYLHEXYL ACRYLATE CROSSPOLYMER COPOLYMER”
Add5: NOF “CERACUTE-F”; INCI name, “GLYCERYLAMIDOETHYL METHACRYLATE/STEARYL METHACRYLATE COPOLYMER”
Add6: Sekisui Kasei “LMX-5C”; INCI name, “METHYL METHACRYLATE CROSSPOLYMER”
Add7: Ashland Japan “UltraThix P-100”; INCI name, “ACRYLIC ACID/VP CROSSPOLYMER”
Add8: Dow Chemical “ACUDYN 1000 Polymer”; INCI name, “ACRYLATES/HYDROXYESTERS ACRYLATES COPOLYMER”
Add9: Dow Chemical “Antaron Sensory”; INCI name, “VP/ACRYLATES/LAURYL METHACRYLATE COPOLYMER”

### Others

Add10: Tokyo Chemical Industry, triethyl citrate
Add11: Kokyu Alcohol Kogyo “KAK-DIBA,” diisobutyl adipate
Add12: Daihachi Chemical Industry “Daifatty101,” a mixture of dibasic acid esters

### Aromatic Compounds (B)

Add13: Cardolite “GX2053,” cardanol
Add31: DIC, 4-octylphenol
Add32: FUJIFILM Wako Pure Chemical, 4-dodecylphenol
Add33: FUJIFILM Wako Pure Chemical, 4-2,2-dibutyl-octylphenol

### Compounds for the First Coating Layer

### Polyamine Compounds

Fir1: Nippon Shokubai “EPOMIN SP-003”; polyethyleneimine; molecular weight, 300
Fir2: Nippon Shokubai “EPOMIN SP-006”; polyethyleneimine; molecular weight, 600
Fir3: Nippon Shokubai “EPOMIN SP-012”; polyethyleneimine; molecular weight, 1200
Fir4: Nippon Shokubai “EPOMIN SP-018”; polyethyleneimine; molecular weight, 1800
Fir5: Nippon Shokubai “EPOMIN SP-200”; polyethyleneimine; molecular weight, 1000
Fir6: Nippon Shokubai “EPOMIN HM-2000”; polyethyleneimine; molecular weight, 30000
Fir7: Nippon Shokubai “EPOMIN P-1000”; polyethyleneimine; molecular weight, 70000
Fir8: Nittobo Medical “PAA-01”; polyallylamine; molecular weight, 1600
Fir9: Nittobo Medical “PAA-03”; polyallylamine; molecular weight, 3000
Fir10: Nittobo Medical “PAA-05”; polyallylamine; molecular weight, 5000
Fir11: Nittobo Medical “PAA-08”; polyallylamine; molecular weight, 8000
Fir12: Nittobo Medical “PAA-15C”; polyallylamine; molecular weight, 15000
Fir13: Nittobo Medical “PAA-25”; polyallylamine; molecular weight, 25000
Fir14: Mitsubishi Chemical “GOHSENOL N-300,” polyvinyl alcohol
Fir15: JNC “Polylysine 10,” polylysine
Fir16: Ichimaru Pharcos “Polylysine 10,” polylysine Polyvinyl Alcohol and Polyvinylpyrrolidone
Fir17: Mitsubishi Chemical “GOHSENOL N-300,” polyvinyl alcohol
Fir18: Nippon Shokubai “K-30,” polyvinylpyrrolidone Linear-Chain Saturated Fatty Acids
Fir19: NOF “NAA-222S,” behenic acid (C22)
Fir20: FUJIFILM Wako Pure Chemical, arachidic acid (C20)
Fir21: FUJIFILM Wako Pure Chemical, palmitic acid (C14)
Fir22: FUJIFILM Wako Pure Chemical, lauric acid (C12)
Fir23: FUJIFILM Wako Pure Chemical, lignoceric acid (C24) Hydroxy Fatty Acids
Fir24: Itoh Oil Chemicals “12-Hydroxystearic Acid,” hydroxystearic acid
Fir25: NOF, a hydrogenated castor oil fatty acid

Amino Acid Compound
Fir26: Ajinomoto “AMIHOPE LL,” lauroyl lysine

### Compounds for the Second Coating Layer

### Waxes

Sec1: Senka “CN-100,” carnauba wax
Sec2: Toa Kasei “TOWAX-1F3,” carnauba wax
Sec3: Toa Kasei “TOWAX-1F6,” carnauba wax
Sec4: Toa Kasei “TOWAX-1F8,” carnauba wax
Sec5: Toa Kasei “TOWAX-1F12,” carnauba wax
Sec6: Toa Kasei “TOWAX-5B2,” carnauba wax
Sec7: Toa Kasei “TOWAX-1B4,” carnauba wax
Sec8: Toa Kasei “TOWAX-4F2,” candelilla wax
Sec9: Toa Kasei “TOWAX-4F3,” candelilla wax
Sec10: Toa Kasei “TOWAX-4F4,” candelilla wax
Sec11: Toa Kasei “TOWAX-6B2,” Rosa damascena flower wax
Sec12: Toa Kasei “TOWAX-6F2,” sunflower seed wax
Sec13: Kokura Gosei Kogyo, rice bran wax
Sec14: Boso Oil and Fat “SS-1,” rice bran wax
Sec15: Nisshin OilliO “COSMOL 222,” diisostearyl malate

### Polyvalent Metal Salts

Sec21: FUJIFILM Wako Pure Chemical, aluminum sulfate
Sec22: FUJIFILM Wako Pure Chemical, polyaluminum chloride
Sec23: FUJIFILM Wako Pure Chemical, iron chloride
Sec24: FUJIFILM Wako Pure Chemical, calcium hydroxide

### External Additives

### Silicon-Containing Compound Particles

Sur1: Nippon Aerosil “AEROSIL R972,” silica dimethyl silylate particles, volume-average particle diameter=16 nm
Sur2: Nippon Aerosil “AEROSIL RY200S,” silica dimethicone silylate particles, volume-average particle diameter=12 nm

### Metallic Soap Particles

Sur3: NOF “MZ-2,” zinc stearate particles, volume-average particle diameter=1.5 μm
Sur4: NOF “Magnesium Stearate S,” magnesium stearate particles, volume-average particle diameter=1 μm

### Fatty Acid Ester Particles

Sur6: Kao “EXCEPARL SS,” stearyl stearate particles, volume-average particle diameter=1 μm

### Metal Oxide Particles

Sur7: Sakai Chemical “FINEX-50,” zinc oxide particles, volume-average particle diameter=1.5 μm

The volume-average particle diameters of the external additives are measured through the same procedure as the volume-average particle diameters of the cellulosic particles.

### Example 1

### Particle Precursor Production Step

Eight hundred parts of Cel1 as a cellulose acylate and 200 parts of Add1 as the second component are kneaded together in a twin-screw kneader (Toshiba Machine, TEX41SS) adjusted to a cylinder temperature of 220° C. to give a resin in pellet form (hereinafter referred to as resin pellets.).

One hundred and thirty parts of the resin pellets are dissolved completely in 870 parts of ethyl acetate. The resulting solution is added to a water-based liquid containing 50 parts of calcium carbonate and 500 parts of purified water, and the resulting mixture is stirred for 3 hours (hereinafter referred to as “the first stirring time”). A dispersion of 4 parts of carboxymethyl cellulose (hereinafter also referred to as “CMC”) and 200 parts of methyl ethyl ketone in 600 parts of purified water is added, and the resulting mixture is stirred for 5 minutes using a high-speed emulsifier. Ten parts of sodium hydroxide is added, and the resulting mixture is heated to 80° C. and stirred for 3 hours to eliminate the ethyl acetate and the methyl ethyl ketone. The same amount of diluted hydrochloric acid as the sodium hydroxide is added, the residue is collected by filtration, and the collected solids are dispersed once again in purified water to give a particle precursor dispersion (solids concentration, 10%).

### Saponification Step

A mixture obtained by adding 17.5 parts of a 20% aqueous solution of sodium hydroxide to 500 parts of the particle precursor dispersion is stirred for 6 hours at a saponification temperature of 30° C. After the pH is adjusted to 7 with hydrochloric acid, the saponified slurry is cleaned by repeated filtration and washing until the electrical conductivity of the filtrate is 10 μs/cm or less, yielding cellulosic particles.

### Examples 2 to 20 and Comparative Examples 1 to 3

### Particle Precursor Production Step

A particle precursor dispersion (solids concentration, 10%) is obtained through the same procedure as in the particle precursor production step in Example 1, except that the cellulose acylate and its amount, the second component and its amount, and the cylinder temperature in the preparation of the resin pellets are as in Table 1.

### Saponification Step

Cellulosic particles are obtained through the same procedure as in the saponification step in Example 1.

### Example 21

### Particle Precursor Production Step

A particle precursor dispersion (solids concentration, 10%) is obtained through the same procedure as in the particle precursor production step in Example 1.

### Saponification Step

Cellulosic particles are obtained through the same procedure as in the saponification step in Example 1.

### Coating Layer Formation Step

One thousand parts of the cellulosic particles, which are core particles, and 10000 parts of deionized water are mixed together to give a core particle dispersion. Five parts of Fir16 as a compound that will form the first coating layer is added to the core particle dispersion, and the resulting mixture is stirred for 1 hour to make the compound form a coating layer. The cellulosic particles having a coating layer are cleaned by repeated filtration and washing until the electrical conductivity of the filtrate is 10 μs/cm or less, yielding cellulosic particles having a coating layer.

### Examples 22 to 38

Cellulosic particles having a coating layer are obtained through the same procedure as in Example 21, except that in the coating layer formation step, the species of the compound that will form the first coating layer (“First-layer compound” in Table 1) is as in Table 1.

### Example 39

### Particle Precursor Production Step

A particle precursor dispersion (solids concentration, 10%) is obtained through the same procedure as in the particle precursor production step in Example 1.

### Saponification Step

Cellulosic particles are obtained through the same procedure as in the saponification step in Example 1.

### Coating Layer Formation Step

One thousand parts of the cellulosic particles, which are core particles, and 10000 parts of deionized water are mixed together to give a core particle dispersion. Five parts of Fin 6 as a compound that will form the first coating layer is added to the core particle dispersion, and the resulting mixture is stirred for 1 hour to make the compound form a first coating layer, yielding a dispersion of cellulosic particles having a first coating layer.

Subsequently, an emulsion for the formation of the second coating layer is prepared by stirring 4 parts of Sec1 as a wax and 50 parts of purified water using a high-speed emulsifier.

All of the emulsion for the formation of the second coating layer is added to the dispersion of cellulosic particles having a first coating layer, and the resulting mixture is stirred for 24 hours to make the wax form the second coating layer, yielding a dispersion of cellulosic particles having first and second coating layers.

The cellulosic particles having first and second coating layers are cleaned by repeated filtration and washing until the electrical conductivity of the filtrate is 10 μs/cm or less, yielding cellulosic particles having first and second coating layers.

### Examples 40 to 56

Cellulosic particles having first and second coating layers are obtained through the same procedure as in Example 39, except that in the coating layer formation step, the amount of the compound that will form the first coating layer, the wax species, and the amount of the wax are as in Table 1.

### Example 57

### Particle Precursor Production Step, Saponification Step, and Coating Layer Formation Step

Cellulosic particles having first and second coating layers are obtained through the same procedure as in Example 39.

### Addition Step

As an external additive, 0.6 parts of Sur1 is added to 30 parts of the cellulosic particles having first and second coating layers, and the ingredients are mixed together in a mixing mill (WONDER CRUSHER, Osaka Chemical) to give cellulosic particles having an external additive.

### Examples 58 to 64

Cellulosic particles having an external additive are obtained through the same procedure as in Example 57, except that in the addition step, the external additive and its amount are as in Table 1.

### Examples 65 to 72

Cellulosic particles having first and second coating layers are obtained through the same procedure as in Example 39, except that in the particle precursor production step, the amount of calcium carbonate, the first stirring time, and the amount of sodium hydroxide are as in Table 1.

### Example 73

Cellulosic particles having a coating layer are obtained through the same procedure as in Example 39, except that the coating layer formation step is done without the process of adding 5 parts of Fir16 as a compound that will form the first coating layer to the core particle dispersion and stirring the resulting mixture for 1 hour.

### Comparative Example 4

### Particle Precursor Production Step

As a cellulose acylate, 130 parts of Cel1 is dissolved completely in 870 parts of ethyl acetate. The resulting solution is added to a water-based liquid containing 55 parts of calcium carbonate and 500 parts of purified water, and the resulting mixture is stirred for 2 hours. A dispersion of 5 parts of carboxymethyl cellulose and 200 parts of methyl ethyl ketone in 600 parts of purified water is added, and the resulting mixture is stirred for 5 minutes using a high-speed emulsifier. Ten parts of sodium hydroxide is added, and the resulting mixture is heated to 80° C. and stirred for 3 hours to eliminate the ethyl acetate and the methyl ethyl ketone. The same amount of diluted hydrochloric acid as the sodium hydroxide is added, the residue is collected by filtration, and the collected solids are dispersed once again in purified water to give a particle precursor dispersion (solids concentration, 10%).

### Saponification Step

Cellulosic particles are obtained through the same procedure as in the saponification step in Example 1.

### Comparative Example 5

### Particle Precursor Production Step and Saponification Step

Cellulosic particles are obtained through the same procedure as in Comparative Example 4.

### Coating Layer Formation Step

Cellulosic particles having a coating layer are obtained through the same procedure as in the coating layer formation step in Example 21, except that the cellulosic particles obtained as described above are used as core particles and that the amount of the compound that will form the first coating layer per 100 parts of the core particles is changed as in Table 1.

### Comparative Example 6

### Particle Precursor Production Step and Saponification Step

Cellulosic particles are obtained through the same procedure as in Comparative Example 4.

### Coating Layer Formation Step

Cellulosic particles having first and second coating layers are obtained through the same procedure as in the coating layer formation step in Example 39, except that the cellulosic particles obtained as described above are used as core particles and that the amount of the compound that will form the first coating layer per 100 parts of the core particles is changed as in Table 1.

### Comparative Example 7

### Particle Precursor Production Step, Saponification Step, and Coating Layer Formation Step

Cellulosic particles having first and second coating layers are obtained through the same procedure as in Comparative Example 6.

Cellulosic particles having an external additive are obtained through the same procedure as in the addition step in Example 57, except that the cellulosic particles having first and second coating layers obtained as described above are used.

### Examples 74 to 77

### Particle Precursor Production Step, Saponification Step, and Coating Layer Formation Step

Cellulosic particles having first and second coating layers are obtained through the same procedure as in Example 39, except that in the coating layer formation step, the wax species is changed as in Table 1 and that in preparing the emulsion for the formation of the second coating layer, the polyvalent metal salt specified in Table 1, its amount being as in Table 1, is added together with the wax and the purified water.

### Addition Step

Cellulosic particles having an external additive are obtained through the same procedure as in the addition step in Example 57, except that the cellulosic particles having first and second coating layers obtained as described above are used.

### Comparative Examples 8 to 12

The following particles are used as the cellulosic particles of each example.

Comparative Example 8: CELLULOBEADS D10 (Daito Kasei)

Comparative Example 9: OTS-0.5A CELLULOBEADS D10 (Daito Kasei)

Comparative Example 10: S-STM CELLULOBEADS D-5 (Daito Kasei)

Comparative Example 11: CELLUFLOW C25 (JNC)

Comparative Example 12: CELLUFLOW TA25 (JNC)

### Comparative Examples 13 to 16

The cellulosic particles of each example are obtained according to the following procedures.

Comparative Example 13: Cellulosic particles are obtained according to the procedure described in Example 1 in Japanese Patent No. 6872068.

Comparative Example 14: Cellulosic particles are obtained according to the procedure described in Example 2 in Japanese Patent No. 6872068.

Comparative Example 15: Cellulosic particles are obtained according to the procedure described in Example 1 in Japanese Unexamined Patent Application Publication No. 2021-021044.

Comparative Example 16: Cellulosic particles are obtained according to the procedure described in Example 1 in Japanese Unexamined Patent Application Publication No. 2021-021045.

### Examples 78 to 82

Cellulosic particles are obtained through the same procedure as in Example 1, except that in the particle precursor production step, the second component is changed as in Table 1.

### Examples 83 to 92

Cellulosic particles having a coating layer are obtained through the same procedure as in Example 21, except that in the coating layer formation step, the compound that will form the first coating layer and its amount are changed as in Table 1.

### Example 93

Cellulosic particles having first and second coating layers are obtained through the same procedure as in Example 39, except that in the coating layer formation step, the compound that will form the first coating layer (“First-layer compound” in Table 1) and its amount are as in Table 1.

### Example 94

Cellulosic particles having first and second coating layers are obtained through the same procedure as in Example 93, except that in preparing the emulsion for the formation of the second coating layer in the coating layer formation step, the polyvalent metal salt specified in Table 1, its amount being as in Table 1, is added together with the wax and the purified water.

### Example 95

### Particle Precursor Production Step, Saponification Step, and Coating Layer Formation Step

Cellulosic particles having first and second coating layers are obtained through the same procedure as in Example 94.

### Addition Step

As an external additive, 0.6 parts of Sur1 is added to 30 parts of the cellulosic particles having first and second coating layers, and the ingredients are mixed together in a mixing mill (WONDER CRUSHER, Osaka Chemical) to give cellulosic particles having an external additive.

### Evaluations

Biodegradability and flexibility evaluations are carried out using the cellulosic particles obtained in each example or comparative example.

### Biodegradability Evaluation

Percentage biodegradation after 60 days is measured and calculated as per JIS K6950:2000 (ISO 14851:1999).

### Flexibility Evaluation

Young's modulus is calculated using a micro-compression tester (MCT-510, Shimadzu). Specifically, the cellulosic particles are sprinkled over the sample stage, and while the stage is monitored with an optical microscope, the initial position is adjusted so that one single particle will come right beneath the tip of the indenter. The particle is compressed at a stage moving speed of 0.2 μm/s, and the test force as a function of displacement is detected continuously. The measurement is ended when the particle breaks completely. The resulting stress-strain curve is represented by two straight lines with different slopes. With the point of intersection between these two straight lines as the yield point (cy, ay), the slope of a straight line drawn between this point and the origin is defined as apparent Young's modulus Ey as in the equation below. Values obtained according to the following equation are presented in Table 2.

Ey=σy/εy Equation:

TABLE 1

| | | Particle precursor production step | | | | | | | |
|---|---|---|---|---|---|---|---|---|---|
| | | | | Second component | | | | Amount of | |
| | | Cellulose acylate | | | Octanol/water | | Cylinder | calcium | First |
| | Particle | | Amount | | partition | Amount | temperature | carbonate | stirring |
| | number | Species | (parts) | Species | coefficient | (parts) | (° C.) | (parts) | time (hr) |
| Example 1 | Par401 | Cel1 | 800 | Add1 | 6 | 200 | 220 | 50 | 3 |
| Example 2 | Par402 | Cel2 | 800 | Add1 | 6 | 200 | 240 | 50 | 3 |
| Example 3 | Par403 | Cel3 | 800 | Add1 | 6 | 200 | 200 | 50 | 3 |
| Example 4 | Par404 | Cel4 | 800 | Add1 | 6 | 200 | 200 | 50 | 3 |
| Example 5 | Par405 | Cel5 | 800 | Add1 | 6 | 200 | 220 | 50 | 3 |
| Example 6 | Par406 | Cel6 | 800 | Add1 | 6 | 200 | 200 | 50 | 3 |
| Example 7 | Par407 | Cel7 | 800 | Add1 | 6 | 200 | 200 | 50 | 3 |
| Example 8 | Par408 | Cel1 | 800 | Add2 | 9 | 200 | 220 | 50 | 3 |
| Example 9 | Par409 | Cel1 | 800 | Add3 | Unmeasurable | 200 | 220 | 50 | 3 |
| Example 10 | Par410 | Cel1 | 800 | Add4 | Unmeasurable | 200 | 220 | 50 | 3 |
| Example 11 | Par412 | Cel1 | 800 | Add5 | Unmeasurable | 200 | 220 | 50 | 3 |
| Example 12 | Par413 | Cel1 | 800 | Add6 | Unmeasurable | 200 | 220 | 50 | 3 |
| Example 13 | Par414 | Cel1 | 800 | Add7 | Unmeasurable | 200 | 220 | 50 | 3 |
| Example 14 | Par415 | Cel1 | 800 | Add8 | Unmeasurable | 200 | 220 | 50 | 3 |
| Example 15 | Par416 | Cel1 | 800 | Add9 | Unmeasurable | 200 | 220 | 50 | 3 |
| Comparative | Par417 | Cel1 | 700 | Add10 | 0.9 | 300 | 220 | 50 | 3 |
| Example 1 | | | | | | | | | |
| Comparative | Par418 | Cel1 | 700 | Add11 | 4 | 300 | 220 | 50 | 3 |
| Example 2 | | | | | | | | | |
| Comparative | Par419 | Cel1 | 700 | Add12 | 5 | 300 | 220 | 50 | 3 |
| Example 3 | | | | | | | | | |
| Example 16 | Par420 | Cel1 | 800 | Add13 | 18 | 200 | 220 | 50 | 3 |
| Example 17 | Par421 | Cel1 | 670 | Add1 | 6 | 330 | 220 | 50 | 3 |
| Example 18 | Par422 | Cel1 | 650 | Add1 | 6 | 350 | 220 | 50 | 3 |
| Example 19 | Par423 | Cel1 | 930 | Add1 | 6 | 70 | 220 | 50 | 3 |
| Example 20 | Par424 | Cel1 | 950 | Add1 | 6 | 50 | 240 | 50 | 3 |
| Example 21 | Par425 | Cel1 | 800 | Add1 | 6 | 200 | 220 | 50 | 3 |
| Example 22 | Par426 | Cel1 | 800 | Add1 | 6 | 200 | 220 | 50 | 3 |
| Example 23 | Par427 | Cel1 | 800 | Add1 | 6 | 200 | 220 | 50 | 3 |
| Example 24 | Par428 | Cel1 | 800 | Add1 | 6 | 200 | 220 | 50 | 3 |
| Example 25 | Par429 | Cel1 | 800 | Add1 | 6 | 200 | 220 | 50 | 3 |
| Example 26 | Par430 | Cel1 | 800 | Add1 | 6 | 200 | 220 | 50 | 3 |
| Example 27 | Par431 | Cel1 | 800 | Add1 | 6 | 200 | 220 | 50 | 3 |
| Example 28 | Par432 | Cel1 | 800 | Add1 | 6 | 200 | 220 | 50 | 3 |
| Example 29 | Par433 | Cel1 | 800 | Add1 | 6 | 200 | 220 | 50 | 3 |
| Example 30 | Par434 | Cel1 | 800 | Add1 | 6 | 200 | 220 | 50 | 3 |
| Example 31 | Par435 | Cel1 | 800 | Add1 | 6 | 200 | 220 | 50 | 3 |
| Example 32 | Par436 | Cel1 | 800 | Add1 | 6 | 200 | 220 | 50 | 3 |
| Example 33 | Par437 | Cel1 | 800 | Add1 | 6 | 200 | 220 | 50 | 3 |
| Example 34 | Par438 | Cel1 | 800 | Add1 | 6 | 200 | 220 | 50 | 3 |
| Example 35 | Par439 | Cel1 | 800 | Add1 | 6 | 200 | 220 | 50 | 3 |
| Example 36 | Par440 | Cel1 | 800 | Add1 | 6 | 200 | 220 | 50 | 3 |
| Example 37 | Par441 | Cel1 | 800 | Add1 | 6 | 200 | 220 | 50 | 3 |
| Example 38 | Par442 | Cel1 | 800 | Add1 | 6 | 200 | 220 | 50 | 3 |
| Example 39 | Par443 | Cel1 | 800 | Add1 | 6 | 200 | 220 | 50 | 3 |
| Example 40 | Par444 | Cel1 | 800 | Add1 | 6 | 200 | 220 | 50 | 3 |
| Example 41 | Par445 | Cel1 | 800 | Add1 | 6 | 200 | 220 | 50 | 3 |
| Example 42 | Par446 | Cel1 | 800 | Add1 | 6 | 200 | 220 | 50 | 3 |
| Example 43 | Par447 | Cel1 | 800 | Add1 | 6 | 200 | 220 | 50 | 3 |
| Example 44 | Par448 | Cel1 | 800 | Add1 | 6 | 200 | 220 | 50 | 3 |
| Example 45 | Par449 | Cel1 | 800 | Add1 | 6 | 200 | 220 | 50 | 3 |
| Example 46 | Par450 | Cel1 | 800 | Add1 | 6 | 200 | 220 | 50 | 3 |
| Example 47 | Par451 | Cel1 | 800 | Add1 | 6 | 200 | 220 | 50 | 3 |
| Example 48 | Par452 | Cel1 | 800 | Add1 | 6 | 200 | 220 | 50 | 3 |
| Example 49 | Par453 | Cel1 | 800 | Add1 | 6 | 200 | 220 | 50 | 3 |
| Example 50 | Par454 | Cel1 | 800 | Add1 | 6 | 200 | 220 | 50 | 3 |
| Example 51 | Par455 | Cel1 | 800 | Add1 | 6 | 200 | 220 | 50 | 3 |
| Example 52 | Par456 | Cel1 | 800 | Add1 | 6 | 200 | 220 | 50 | 3 |
| Example 53 | Par457 | Cel1 | 800 | Add1 | 6 | 200 | 220 | 50 | 3 |
| Example 54 | Par458 | Cel1 | 800 | Add1 | 6 | 200 | 220 | 50 | 3 |
| Example 55 | Par459 | Cel1 | 800 | Add1 | 6 | 200 | 220 | 50 | 3 |
| Example 56 | Par460 | Cel1 | 800 | Add1 | 6 | 200 | 220 | 50 | 3 |
| Example 57 | Par461 | Cel1 | 800 | Add1 | 6 | 200 | 220 | 50 | 3 |
| Example 58 | Par462 | Cel1 | 800 | Add1 | 6 | 200 | 220 | 50 | 3 |
| Example 59 | Par463 | Cel1 | 800 | Add1 | 6 | 200 | 220 | 50 | 3 |
| Example 60 | Par464 | Cel1 | 800 | Add1 | 6 | 200 | 220 | 50 | 3 |
| Example 61 | Par466 | Cel1 | 800 | Add1 | 6 | 200 | 220 | 50 | 3 |
| Example 62 | Par467 | Cel1 | 800 | Add1 | 6 | 200 | 220 | 50 | 3 |
| Example 63 | Par468 | Cel1 | 800 | Add1 | 6 | 200 | 220 | 50 | 3 |
| Example 64 | Par469 | Cel1 | 800 | Add1 | 6 | 200 | 220 | 50 | 3 |
| Example 65 | Par470 | Cel1 | 800 | Add1 | 6 | 200 | 220 | 50 | 1.5 |
| Example 66 | Par471 | Cel1 | 800 | Add1 | 6 | 200 | 220 | 50 | 1 |
| Example 67 | Par472 | Cel1 | 800 | Add1 | 6 | 200 | 220 | 65 | 3 |
| Example 68 | Par473 | Cel1 | 800 | Add1 | 6 | 200 | 220 | 70 | 3 |
| Example 69 | Par474 | Cel1 | 800 | Add1 | 6 | 200 | 220 | 40 | 3 |
| Example 70 | Par475 | Cel1 | 800 | Add1 | 6 | 200 | 220 | 35 | 3 |
| Example 71 | Par476 | Cel1 | 800 | Add1 | 6 | 200 | 220 | 50 | 3 |
| Example 72 | Par477 | Cel1 | 800 | Add1 | 6 | 200 | 220 | 50 | 3 |
| Example 73 | Par480 | Cel1 | 800 | Add1 | 6 | 200 | 220 | 50 | 3 |
| Comparative | Par481 | Cel1 | | | | | | 55 | 2 |
| Example 4 | | | | | | | | | |
| Comparative | Par482 | Cel1 | | | | | | 55 | 2 |
| Example 5 | | | | | | | | | |
| Comparative | Par483 | Cel1 | | | | | | 55 | 2 |
| Example 6 | | | | | | | | | |
| Comparative | Par484 | Cel1 | | | | | | 55 | 2 |
| Example 7 | | | | | | | | | |
| Example 74 | Par485 | Cel1 | 800 | Add1 | 6 | 200 | 220 | 50 | 3 |
| Example 75 | Par486 | Cel1 | 800 | Add1 | 6 | 200 | 220 | 50 | 3 |
| Example 76 | Par487 | Cel1 | 800 | Add1 | 6 | 200 | 220 | 50 | 3 |
| Example 77 | Par488 | Cel1 | 800 | Add1 | 6 | 200 | 220 | 50 | 3 |
| Example 78 | Par489 | Cel1 | 800 | Add22 | 4 | 200 | 220 | 50 | 3 |
| Example 79 | Par490 | Cel1 | 800 | Add21 | 11 | 200 | 220 | 50 | 3 |
| Example 80 | Par491 | Cel1 | 800 | Add31 | 4 | 200 | 220 | 50 | 3 |
| Example 81 | Par492 | Cel1 | 800 | Add32 | 5 | 200 | 220 | 50 | 3 |
| Example 82 | Par493 | Cel1 | 800 | Add33 | 21 | 200 | 220 | 50 | 3 |
| Example 83 | Par1401 | Cel1 | 800 | Add1 | 6 | 200 | 220 | 50 | 3 |
| Example 84 | Par1402 | Cel1 | 800 | Add1 | 6 | 200 | 220 | 50 | 3 |
| Example 85 | Par1403 | Cel1 | 800 | Add1 | 6 | 200 | 220 | 50 | 3 |
| Example 86 | Par1404 | Cel1 | 800 | Add1 | 6 | 200 | 220 | 50 | 3 |
| Example 87 | Par1405 | Cel1 | 800 | Add1 | 6 | 200 | 220 | 50 | 3 |
| Example 88 | Par1406 | Cel1 | 800 | Add1 | 6 | 200 | 220 | 50 | 3 |
| Example 89 | Par1407 | Cel1 | 800 | Add1 | 6 | 200 | 220 | 50 | 3 |
| Example 90 | Par1408 | Cel1 | 800 | Add1 | 6 | 200 | 220 | 50 | 3 |
| Example 91 | Par1409 | Cel1 | 800 | Add1 | 6 | 200 | 220 | 50 | 3 |
| Example 92 | Par1410 | Cel1 | 800 | Add1 | 6 | 200 | 220 | 50 | 3 |
| Example 93 | Par1411 | Cel1 | 800 | Add1 | 6 | 200 | 220 | 50 | 3 |
| Example 94 | Par1412 | Cel1 | 800 | Add1 | 6 | 200 | 220 | 50 | 3 |
| Example 95 | Par1413 | Cel1 | 800 | Add1 | 6 | 200 | 220 | 50 | 3 |

| | Particle precursor | | Coating layer | | | | | | | |
|---|---|---|---|---|---|---|---|---|---|---|
| | production step | | formation step | | | | | | | |
| | | Amount of | First-layer | | | | Polyvalent | | Addition step | |
| | Amount | sodium | compound | | Wax | | metal salt | | External additive | |
| | of CMC | hydroxide | | Amount | | Amount | | Amount | | Amount |
| | (parts) | (parts) | Species | (parts) | Species | (parts) | Species | (parts) | Species | (parts) |
| Example 1 | 4 | 10 | | | | | | | | |
| Example 2 | 4 | 10 | | | | | | | | |
| Example 3 | 4 | 10 | | | | | | | | |
| Example 4 | 4 | 10 | | | | | | | | |
| Example 5 | 4 | 10 | | | | | | | | |
| Example 6 | 4 | 10 | | | | | | | | |
| Example 7 | 4 | 10 | | | | | | | | |
| Example 8 | 4 | 10 | | | | | | | | |
| Example 9 | 4 | 10 | | | | | | | | |
| Example 10 | 4 | 10 | | | | | | | | |
| Example 11 | 4 | 10 | | | | | | | | |
| Example 12 | 4 | 10 | | | | | | | | |
| Example 13 | 4 | 10 | | | | | | | | |
| Example 14 | 4 | 10 | | | | | | | | |
| Example 15 | 4 | 10 | | | | | | | | |
| Comparative | 4 | 10 | | | | | | | | |
| Example 1 | | | | | | | | | | |
| Comparative | 4 | 10 | | | | | | | | |
| Example 2 | | | | | | | | | | |
| Comparative | 4 | 10 | | | | | | | | |
| Example 3 | | | | | | | | | | |
| Example 16 | 4 | 10 | | | | | | | | |
| Example 17 | 4 | 10 | | | | | | | | |
| Example 18 | 4 | 10 | | | | | | | | |
| Example 19 | 4 | 10 | | | | | | | | |
| Example 20 | 4 | 10 | | | | | | | | |
| Example 21 | 4 | 10 | Fir16 | 5 | | | | | | |
| Example 22 | 4 | 10 | Fir1 | 5 | | | | | | |
| Example 23 | 4 | 10 | Fir2 | 5 | | | | | | |
| Example 24 | 4 | 10 | Fir3 | 5 | | | | | | |
| Example 25 | 4 | 10 | Fir4 | 5 | | | | | | |
| Example 26 | 4 | 10 | Fir5 | 5 | | | | | | |
| Example 27 | 4 | 10 | Fir6 | 5 | | | | | | |
| Example 28 | 4 | 10 | Fir7 | 5 | | | | | | |
| Example 29 | 4 | 10 | Fir8 | 5 | | | | | | |
| Example 30 | 4 | 10 | Fir9 | 5 | | | | | | |
| Example 31 | 4 | 10 | Fir10 | 5 | | | | | | |
| Example 32 | 4 | 10 | Fir11 | 5 | | | | | | |
| Example 33 | 4 | 10 | Fir12 | 5 | | | | | | |
| Example 34 | 4 | 10 | Fir13 | 5 | | | | | | |
| Example 35 | 4 | 10 | Fir14 | 5 | | | | | | |
| Example 36 | 4 | 10 | Fir15 | 5 | | | | | | |
| Example 37 | 4 | 10 | Fir17 | 5 | | | | | | |
| Example 38 | 4 | 10 | Fir18 | 5 | | | | | | |
| Example 39 | 4 | 10 | Fir16 | 5 | Sec1 | 4 | | | | |
| Example 40 | 4 | 10 | Fir16 | 5 | Sec2 | 4 | | | | |
| Example 41 | 4 | 10 | Fir16 | 5 | Sec3 | 4 | | | | |
| Example 42 | 4 | 10 | Fir16 | 5 | Sec4 | 4 | | | | |
| Example 43 | 4 | 10 | Fir16 | 5 | Sec5 | 4 | | | | |
| Example 44 | 4 | 10 | Fir16 | 5 | Sec6 | 4 | | | | |
| Example 45 | 4 | 10 | Fir16 | 5 | Sec7 | 4 | | | | |
| Example 46 | 4 | 10 | Fir16 | 5 | Sec8 | 4 | | | | |
| Example 47 | 4 | 10 | Fir16 | 5 | Sec9 | 4 | | | | |
| Example 48 | 4 | 10 | Fir16 | 5 | Sec10 | 4 | | | | |
| Example 49 | 4 | 10 | Fir16 | 5 | Sec11 | 4 | | | | |
| Example 50 | 4 | 10 | Fir16 | 5 | Sec12 | 4 | | | | |
| Example 51 | 4 | 10 | Fir16 | 5 | Sec13 | 4 | | | | |
| Example 52 | 4 | 10 | Fir16 | 5 | Sec14 | 4 | | | | |
| Example 53 | 4 | 10 | Fir16 | 5 | Sec15 | 4 | | | | |
| Example 54 | 4 | 10 | Fir16 | 10 | Sec1 | 2 | | | | |
| Example 55 | 4 | 10 | Fir16 | 5 | Sec1 | 8 | | | | |
| Example 56 | 4 | 10 | Fir16 | 10 | Sec1 | 8 | | | | |
| Example 57 | 4 | 10 | Fir16 | 5 | Sec1 | 4 | | | Sur1 | 0.6 |
| Example 58 | 4 | 10 | Fir16 | 5 | Sec1 | 4 | | | Sur2 | 0.6 |
| Example 59 | 4 | 10 | Fir16 | 5 | Sec1 | 4 | | | Sur3 | 0.6 |
| Example 60 | 4 | 10 | Fir16 | 5 | Sec1 | 4 | | | Sur4 | 0.6 |
| Example 61 | 4 | 10 | Fir16 | 5 | Sec1 | 4 | | | Sur6 | 0.6 |
| Example 62 | 4 | 10 | Fir16 | 5 | Sec1 | 4 | | | Sur7 | 0.6 |
| Example 63 | 4 | 10 | Fir16 | 5 | Sec1 | 4 | | | Sur1 | 0.3 |
| Example 64 | 4 | 10 | Fir16 | 5 | Sec1 | 4 | | | Sur1 | 0.9 |
| Example 65 | 4 | 10 | Fir16 | 5 | Sec1 | 4 | | | | |
| Example 66 | 4 | 10 | Fir16 | 5 | Sec1 | 4 | | | | |
| Example 67 | 4 | 10 | Fir16 | 5 | Sec1 | 4 | | | | |
| Example 68 | 4 | 10 | Fir16 | 5 | Sec1 | 4 | | | | |
| Example 69 | 4 | 10 | Fir16 | 5 | Sec1 | 4 | | | | |
| Example 70 | 4 | 10 | Fir16 | 5 | Sec1 | 4 | | | | |
| Example 71 | 4 | 7 | Fir16 | 5 | Sec1 | 4 | | | | |
| Example 72 | 4 | 5 | Fir16 | 5 | Sec1 | 4 | | | | |
| Example 73 | 4 | 10 | | | Sec1 | 4 | | | | |
| Comparative | 5 | 10 | | | | | | | | |
| Example 4 | | | | | | | | | | |
| Comparative | 5 | 10 | Fir16 | 10 | | | | | | |
| Example 5 | | | | | | | | | | |
| Comparative | 5 | 10 | Fir16 | 10 | Sec1 | 4 | | | | |
| Example 6 | | | | | | | | | | |
| Comparative | 5 | 10 | Fir16 | 10 | Sec1 | 4 | | | Sur1 | 0.6 |
| Example 7 | | | | | | | | | | |
| Example 74 | 4 | 10 | Fir16 | 5 | Sec3 | 4 | Sec21 | 0.012 | Sur1 | 0.6 |
| Example 75 | 4 | 10 | Fir16 | 5 | Sec3 | 4 | Sec22 | 0.012 | Sur1 | 0.6 |
| Example 76 | 4 | 10 | Fir16 | 5 | Sec3 | 4 | Sec23 | 0.012 | Sur1 | 0.6 |
| Example 77 | 4 | 10 | Fir16 | 5 | Sec3 | 4 | Sec24 | 0.012 | Sur1 | 0.6 |
| Example 78 | 4 | 10 | | | | | | | | |
| Example 79 | 4 | 10 | | | | | | | | |
| Example 80 | 4 | 10 | | | | | | | | |
| Example 81 | 4 | 10 | | | | | | | | |
| Example 82 | 4 | 10 | | | | | | | | |
| Example 83 | 4 | 10 | Fir19 | 8 | | | | | | |
| Example 84 | 4 | 10 | Fir20 | 8 | | | | | | |
| Example 85 | 4 | 10 | Fir21 | 8 | | | | | | |
| Example 86 | 4 | 10 | Fir22 | 8 | | | | | | |
| Example 87 | 4 | 10 | Fir23 | 8 | | | | | | |
| Example 88 | 4 | 10 | Fir24 | 8 | | | | | | |
| Example 89 | 4 | 10 | Fir25 | 8 | | | | | | |
| Example 90 | 4 | 10 | Fir26 | 8 | | | | | | |
| Example 91 | 4 | 10 | Fir19 | 6 | | | | | | |
| Example 92 | 4 | 10 | Fir19 | 10 | | | | | | |
| Example 93 | 4 | 10 | Fir19 | 8 | Sec1 | 4 | | | | |
| Example 94 | 4 | 10 | Fir19 | 8 | Sec1 | 4 | Sec21 | 0.012 | | |
| Example 95 | 4 | 10 | Fir19 | 8 | Sec1 | 4 | Sec21 | 0.012 | Sur1 | 0.6 |

TABLE 2

| | | Particle characteristics | | | | | | | | |
|---|---|---|---|---|---|---|---|---|---|---|
| | | | | Number- | | | | | | |
| | | | | average | | | | | | |
| | | Amount | Amount | particle | | | Number- | | | Flexibility |
| | | of the | of the | diameter of | | | average | Surface | Biodegradability | Young's |
| | | first | second | cellulosic | | | molecular | smooth- | Percentage | modulus |
| | Particle | component | component | particles | | | weight of | ness | biodegradation | Ey |
| | number | (parts) | (parts) | (μm) | GSDv | Sphericity | cellulose | (%) | (%) | (MPa) |
| Example 1 | Par401 | 82 | 18 | 8 | 1.12 | 0.98 | 46000 | 96 | 74 | 18 |
| Example 2 | Par402 | 82 | 18 | 7 | 1.12 | 0.96 | 59000 | 97 | 72 | 17 |
| Example 3 | Par403 | 81 | 19 | 8 | 1.31 | 0.95 | 73000 | 95 | 68 | 24 |
| Example 4 | Par404 | 82 | 18 | 6 | 1.41 | 0.96 | 49000 | 96 | 66 | 26 |
| Example 5 | Par405 | 80 | 20 | 8 | 1.33 | 0.94 | 36000 | 88 | 68 | 35 |
| Example 6 | Par406 | 80 | 20 | 7 | 1.32 | 0.95 | 23000 | 85 | 68 | 33 |
| Example 7 | Par407 | 80 | 20 | 6 | 1.24 | 0.98 | 12000 | 83 | 68 | 37 |
| Example 8 | Par408 | 82 | 18 | 7 | 1.25 | 0.97 | 46000 | 95 | 68 | 18 |
| Example 9 | Par409 | 82 | 18 | 8 | 1.33 | 0.98 | 47000 | 93 | 66 | 25 |
| Example 10 | Par410 | 83 | 17 | 6 | 1.32 | 0.98 | 46000 | 95 | 65 | 26 |
| Example 11 | Par412 | 82 | 18 | 7 | 1.28 | 0.97 | 46000 | 96 | 66 | 25 |
| Example 12 | Par413 | 82 | 18 | 8 | 1.32 | 0.98 | 45000 | 97 | 68 | 28 |
| Example 13 | Par414 | 82 | 18 | 7 | 1.29 | 0.97 | 46000 | 96 | 68 | 25 |
| Example 14 | Par415 | 83 | 17 | 6 | 1.36 | 0.98 | 47000 | 95 | 65 | 24 |
| Example 15 | Par416 | 83 | 17 | 8 | 1.38 | 0.97 | 46000 | 96 | 65 | 22 |
| Comparative | Par417 | 89 | 11 | 7 | 1.27 | 0.98 | 45000 | 88 | 79 | 125 |
| Example 1 | | | | | | | | | | |
| Comparative | Par418 | 87 | 13 | 6 | 1.33 | 0.98 | 45000 | 89 | 25 | 138 |
| Example 2 | | | | | | | | | | |
| Comparative | Par419 | 88 | 12 | 7 | 1.35 | 0.97 | 46000 | 88 | 24 | 127 |
| Example 3 | | | | | | | | | | |
| Example 16 | Par420 | 81 | 19 | 7 | 1.21 | 0.98 | 46000 | 97 | 68 | 16 |
| Example 17 | Par421 | 72 | 28 | 8 | 1.44 | 0.98 | 47000 | 96 | 66 | 22 |
| Example 18 | Par422 | 68 | 32 | 7 | 1.45 | 0.99 | 46000 | 93 | 61 | 20 |
| Example 19 | Par423 | 95 | 5 | 6 | 1.28 | 0.97 | 45000 | 95 | 70 | 22 |
| Example 20 | Par424 | 96 | 4 | 7 | 1.18 | 0.98 | 46000 | 96 | 68 | 38 |
| Example 21 | Par425 | 82 | 18 | 8 | 1.23 | 0.99 | 47000 | 98 | 68 | 16 |
| Example 22 | Par426 | 83 | 17 | 8 | 1.28 | 0.95 | 45000 | 95 | 66 | 22 |
| Example 23 | Par427 | 81 | 19 | 7 | 1.31 | 0.98 | 48000 | 96 | 65 | 24 |
| Example 24 | Par428 | 82 | 18 | 6 | 1.29 | 0.97 | 46000 | 96 | 66 | 22 |
| Example 25 | Par429 | 82 | 18 | 8 | 1.33 | 0.96 | 43000 | 95 | 67 | 22 |
| Example 26 | Par430 | 83 | 17 | 7 | 1.34 | 0.96 | 47000 | 97 | 66 | 22 |
| Example 27 | Par431 | 82 | 18 | 6 | 1.28 | 0.98 | 47000 | 96 | 68 | 23 |
| Example 28 | Par432 | 81 | 1 | 7 | 1.31 | 0.97 | 46000 | 95 | 67 | 24 |
| Example 29 | Par433 | 82 | 18 | 8 | 1.27 | 0.96 | 47000 | 96 | 68 | 23 |
| Example 30 | Par434 | 83 | 17 | 6 | 1.29 | 0.95 | 48000 | 95 | 65 | 22 |
| Example 31 | Par435 | 83 | 17 | 7 | 1.35 | 0.98 | 47000 | 96 | 66 | 25 |
| Example 32 | Par436 | 82 | 18 | 8 | 1.28 | 0.97 | 47000 | 95 | 68 | 23 |
| Example 33 | Par437 | 82 | 18 | 7 | 1.33 | 0.95 | 45000 | 97 | 67 | 22 |
| Example 34 | Par438 | 81 | 19 | 6 | 1.45 | 0.96 | 47000 | 95 | 68 | 23 |
| Example 35 | Par439 | 83 | 17 | 8 | 1.38 | 0.97 | 45000 | 94 | 65 | 25 |
| Example 36 | Par440 | 82 | 18 | 7 | 1.35 | 0.96 | 47000 | 96 | 66 | 23 |
| Example 37 | Par441 | 82 | 18 | 6 | 1.36 | 0.98 | 47000 | 90 | 66 | 35 |
| Example 38 | Par442 | 83 | 17 | 8 | 1.41 | 0.97 | 48000 | 90 | 67 | 33 |
| Example 39 | Par443 | 82 | 18 | 7 | 1.18 | 0.98 | 47000 | 98 | 66 | 18 |
| Example 40 | Par444 | 83 | 17 | 7 | 1.29 | 0.99 | 47000 | 97 | 66 | 17 |
| Example 41 | Par445 | 82 | 18 | 7 | 1.46 | 0.96 | 47000 | 96 | 66 | 26 |
| Example 42 | Par446 | 82 | 18 | 8 | 1.35 | 0.98 | 48000 | 95 | 67 | 25 |
| Example 43 | Par447 | 83 | 17 | 7 | 1.36 | 0.98 | 47000 | 94 | 68 | 26 |
| Example 44 | Par448 | 82 | 18 | 8 | 1.32 | 0.96 | 47000 | 96 | 68 | 27 |
| Example 45 | Par449 | 83 | 17 | 7 | 1.28 | 0.98 | 46000 | 93 | 67 | 26 |
| Example 46 | Par450 | 83 | 17 | 6 | 1.38 | 0.96 | 43000 | 92 | 68 | 25 |
| Example 47 | Par451 | 84 | 16 | 7 | 1.41 | 0.98 | 47000 | 95 | 67 | 26 |
| Example 48 | Par452 | 82 | 18 | 8 | 1.37 | 0.98 | 47000 | 93 | 66 | 25 |
| Example 49 | Par453 | 83 | 17 | 8 | 1.36 | 0.98 | 45000 | 93 | 65 | 26 |
| Example 50 | Par454 | 82 | 18 | 7 | 1.34 | 0.97 | 47000 | 95 | 66 | 25 |
| Example 51 | Par455 | 83 | 17 | 6 | 1.36 | 0.98 | 47000 | 93 | 68 | 26 |
| Example 52 | Par456 | 83 | 17 | 7 | 1.37 | 0.96 | 47000 | 96 | 67 | 27 |
| Example 53 | Par457 | 82 | 18 | 8 | 1.38 | 0.98 | 46000 | 88 | 61 | 33 |
| Example 54 | Par458 | 82 | 18 | 7 | 1.35 | 0.99 | 47000 | 96 | 66 | 25 |
| Example 55 | Par459 | 82 | 18 | 7 | 1.55 | 0.98 | 48000 | 95 | 67 | 24 |
| Example 56 | Par460 | 82 | 18 | 8 | 1.58 | 0.96 | 47000 | 97 | 6 | 26 |
| Example 57 | Par461 | 82 | 18 | 6 | 1.19 | 0.98 | 47000 | 98 | 65 | 19 |
| Example 58 | Par462 | 83 | 17 | 8 | 1.24 | 0.99 | 44000 | 98 | 65 | 19 |
| Example 59 | Par463 | 82 | 18 | 7 | 1.22 | 0.96 | 45000 | 96 | 65 | 25 |
| Example 60 | Par464 | 82 | 18 | 8 | 1.31 | 0.96 | 47000 | 95 | 65 | 26 |
| Example 61 | Par466 | 83 | 17 | 7 | 1.28 | 0.98 | 45000 | 95 | 65 | 34 |
| Example 62 | Par467 | 82 | 18 | 8 | 1.26 | 0.98 | 47000 | 89 | 65 | 33 |
| Example 63 | Par468 | 82 | 18 | 7 | 1.38 | 0.97 | 47000 | 95 | 66 | 25 |
| Example 64 | Par469 | 83 | 17 | 8 | 1.24 | 0.98 | 46000 | 95 | 65 | 26 |
| Example 65 | Par470 | 00 | 18 | 7 | 1.67 | 0.98 | 47000 | 97 | 66 | 26 |
| Example 66 | Par471 | 83 | 17 | 8 | 1.72 | 0.97 | 48000 | 95 | 65 | 34 |
| Example 67 | Par472 | 83 | 17 | 3 | 1.53 | 0.98 | 47000 | 94 | 68 | 27 |
| Example 68 | Par473 | 83 | 17 | 2 | 1.55 | 0.98 | 47000 | 90 | 69 | 38 |
| Example 69 | Par474 | 83 | 17 | 9 | 1.44 | 0.97 | 47000 | 96 | 66 | 28 |
| Example 70 | Par475 | 82 | 18 | 11 | 1.46 | 0.98 | 47000 | 88 | 65 | 37 |
| Example 71 | Par476 | 82 | 18 | 8 | 1.46 | 0.91 | 45000 | 95 | 66 | 25 |
| Example 72 | Par477 | 83 | 17 | 7 | 1.46 | 0.85 | 47000 | 89 | 66 | 38 |
| Example 73 | Par480 | 82 | 18 | 8 | 1.51 | 0.96 | 46000 | 93 | 68 | 24 |
| Comparative | Par481 | 100 | 0 | 8 | 1.19 | 0.98 | 46000 | 90 | 78 | 110 |
| Example 4 | | | | | | | | | | |
| Comparative | Par482 | 100 | 0 | 7 | 1.23 | 0.99 | 47000 | 90 | 20 | 55 |
| Example 5 | | | | | | | | | | |
| Comparative | Par483 | 100 | 0 | 8 | 1.38 | 0.98 | 46000 | 91 | 80 | 118 |
| Example 6 | | | | | | | | | | |
| Comparative | Par484 | 100 | 0 | 7 | 1.45 | 0.97 | 46000 | 92 | 80 | 113 |
| Example 7 | | | | | | | | | | |
| Example 74 | Par485 | 83 | 17 | 7 | 1.41 | 0.98 | 46000 | 95 | 65 | 18 |
| Example 75 | Par486 | 82 | 18 | 8 | 1.4 | 0.98 | 47000 | 96 | 65 | 19 |
| Example 76 | Par487 | 83 | 17 | 8 | 1.34 | 0.98 | 46000 | 97 | 65 | 18 |
| Example 77 | Par488 | 85 | 15 | 7 | 1.37 | 0.98 | 46000 | 95 | 65 | 19 |
| Comparative | Par101 | 100 | 0 | 14 | 1.17 | 0.97 | 110000 | 96 | 78 | 113 |
| Example 8 | | | | | | | | | | |
| Comparative | Par102 | 100 | 0 | 14 | 1.32 | 0.98 | 110000 | 85 | 79 | 112 |
| Example 9 | | | | | | | | | | |
| Comparative | Par103 | 100 | 0 | 12 | 1.47 | 0.55 | 110000 | 72 | 84 | 115 |
| Example 10 | | | | | | | | | | |
| Comparative | Par104 | 100 | 0 | 10 | 1.86 | 0.97 | 45000 | 80 | 55 | 35 |
| Example 11 | | | | | | | | | | |
| Comparative | Par105 | 0 | 0 | 12 | 1.94 | 0.98 | 48000 | 78 | 76 | 45 |
| Example 12 | | | | | | | | | | |
| Comparative | Par111 | 100 | 0 | 9 | 1.55 | 0.96 | 33000 | 94 | 68 | 48 |
| Example 13 | | | | | | | | | | |
| Comparative | Par112 | 100 | 0 | 9 | 1.62 | 0.96 | 33000 | 95 | 72 | 100 |
| Example 14 | | | | | | | | | | |
| Comparative | Par113 | 100 | 0 | 4 | 1.87 | 0.95 | 44000 | 82 | 75 | 98 |
| Example 15 | | | | | | | | | | |
| Comparative | Par114 | 100 | 0 | 8.2 | 1.88 | 0.96 | 45000 | 80 | 73 | 99 |
| Example 16 | | | | | | | | | | |
| Example 78 | Par489 | 82 | 18 | 7 | 1.35 | 0.96 | 46000 | 95 | 63 | 33 |
| Example 79 | Par490 | 82 | 18 | 7 | 1.38 | 0.97 | 47000 | 93 | 62 | 32 |
| Example 80 | Par491 | 82 | 18 | 7 | 1.4 | 0.98 | 46000 | 95 | 63 | 33 |
| Example 81 | Par492 | 82 | 18 | 8 | 1.39 | 0.97 | 45000 | 95 | 78 | 20 |
| Example 82 | Par493 | 82 | 18 | 7 | 1.44 | 0.98 | 45000 | 93 | 63 | 35 |
| Example 83 | Par1401 | 82 | 18 | 7 | 1.41 | 0.95 | 46000 | 93 | 72 | 19 |
| Example 84 | Par1402 | 82 | 18 | 8 | 1.45 | 0.96 | 46000 | 92 | 66 | 24 |
| Example 85 | Par1403 | 82 | 18 | 7 | 1.44 | 0.96 | 45000 | 92 | 65 | 25 |
| Example 86 | Par1404 | 82 | 18 | 6 | 1.44 | 0.96 | 46000 | 94 | 70 | 32 |
| Example 87 | Par1405 | 82 | 18 | 7 | 1.5 | 0.91 | 46000 | 88 | 61 | 22 |
| Example 88 | Par1406 | 82 | 18 | 8 | 1.45 | 0.93 | 46000 | 90 | 65 | 20 |
| Example 89 | Par1407 | 82 | 18 | 7 | 1.43 | 0.92 | 46000 | 90 | 66 | 20 |
| Example 90 | Par1408 | 82 | 18 | 7 | 1.42 | 0.93 | 45000 | 93 | 70 | 21 |
| Example 91 | Par1409 | 82 | 18 | 6 | 1.45 | 0.94 | 46000 | 92 | 70 | 22 |
| Example 92 | Par1410 | 82 | 18 | 8 | 1.47 | 0.94 | 45000 | 92 | 70 | 24 |
| Example 93 | Par 141 | 82 | 18 | 7 | 1.48 | 0.95 | 46000 | 92 | 71 | 23 |
| Example 94 | Par1412 | 82 | 18 | 8 | 1.5 | 0.95 | 46000 | 91 | 71 | 22 |
| Example 95 | Par1413 | 82 | 18 | 6 | 1.51 | 0.95 | 46000 | 90 | 70 | 21 |

The amounts of the first and second components in Table 2 are calculated as follows.

Ten grams of cellulosic particles having no coating layer and no external additive are put into 500 g of tetrahydrofuran, the resulting mixture is stirred at 50° C. for 4 hours, and then the cellulosic particles are collected by filtration. The collected cellulosic particles are dried at 40° C. for 8 hours, then the mass Wp (g) of the dried particles is measured, and the amount of the first component (unit, “parts”) and that of the second component (unit, “parts”) are determined according to equations (1-1) and (1-2), respectively.

Amount of the first component=(Wp/10)×100 (1-1)

Amount of the second component=((10−Wp)/10)×100 (1-2)

These results indicate that the cellulosic particles according to the Examples may be superior in biodegradability and flexibility.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A cellulosic particle comprising:
a first component that is cellulose; and
a second component that is at least one selected from the group consisting of a fatty acid derivative (A), an aromatic compound having a long-chain aliphatic group and at least one of a phenolic hydroxyl group or a monoglycidyl ether group directly bound to an aromatic group (B), and a (meth)acrylic compound (C).

2. The cellulosic particle according to claim 1, wherein an amount of the first component is 70% by mass or more and 95% by mass or less of a total amount of the first and second components.

3. The cellulosic particle according to claim 1, wherein a total amount of the second component is 5% by mass or more and 30% by mass or less of a total amount of the first and second components.

4. The cellulosic particle according to claim 1, wherein the fatty acid derivative (A) is a fatty acid derivative having a saturated aliphatic group with 10 or more and 25 or fewer carbon atoms.

5. The cellulosic particle according to claim 4, wherein an octanol/water partition coefficient of the fatty acid derivative having a saturated aliphatic group with 10 or more and 25 or fewer carbon atoms is 5 or greater and 10 or less.

6. The cellulosic particle according to claim 1, wherein the fatty acid derivative (A) is a fatty acid ethanolamide.

7. The cellulosic particle according to claim 1, wherein the aromatic compound (B) is an aromatic compound having an aliphatic group with 8 or more and 20 or fewer carbon atoms and at least one of a phenolic hydroxyl group or a monoglycidyl ether group directly bound to an aromatic group (B0).

8. The cellulosic particle according to claim 7, wherein an octanol/water partition coefficient of the aromatic compound (B0) is 5 or greater and 20 or less.

9. The cellulosic particle according to claim 1, comprising:
a core particle containing the first and second components; and
a coating layer covering the core particle and containing at least one selected from the group consisting of a polyamine compound, a wax, a linear-chain saturated fatty acid, a hydroxy fatty acid, and an amino acid compound.

10. The cellulosic particle according to claim 9, wherein the polyamine compound is at least one selected from the group consisting of polyethyleneimine and polylysine.

11. The cellulosic particle according to claim 9, wherein the wax is carnauba wax.

12. The cellulosic particle according to claim 9, wherein the coating layer has a first coating layer covering the core particle and containing the polyamine compound and a second coating layer covering the first coating layer and containing the wax.

13. The cellulosic particle according to claim 12, wherein the second coating layer further contains a polyvalent metal salt.

14. The cellulosic particle according to claim 1, further comprising at least one external additive selected from the group consisting of a silicon-containing compound particle and a metallic soap particle.

15. The cellulosic particle according to claim 14, wherein the silicon-containing compound particle is a silica particle.

16. The cellulosic particle according to claim 1, wherein a volume-average particle diameter of the cellulosic particles is 3 μm or more and less than 10 μm.

17. The cellulosic particle according to claim 1, wherein an upper geometric standard deviation by number GSDv of the cellulosic particles is 1.0 or greater and 1.7 or less.

18. The cellulosic particle according to claim 1, wherein sphericity of the cellulosic particle is 0.90 or greater.

19. The cellulosic particle according to claim 1, wherein a number-average molecular weight of the cellulose is 37000 or more.

20. The cellulosic particle according to claim 1, wherein surface smoothness of the cellulosic particle is 80% or higher.
